(19) United States
(12) Patent Application Publication
(10) Pub. No.: US 2023/0061728 A1
(43) Pub. Date: Mar. 2, 2023

(54) NEUROVASCULAR ACCESS CATHETER WITH MICROCATHETER SEGMENT

(71) Applicant: Imperative Care, Inc., Campbell, CA (US)

(72) Inventors: Daniel Davis, Mill Valley, CA (US); Brandon Yee, Oakland, CA (US); Ashoor Shahbazi Yourgenlow, San Jose, CA (US); Yi Yang, San Francisco, CA (US)

(21) Appl. No.: 17/823,653

(22) Filed: Aug. 31, 2022

Related U.S. Application Data
(63) Continuation-in-part of application No. 17/822,933, filed on Aug. 29, 2022.
(60) Provisional application No. 63/239,256, filed on Aug. 31, 2021.

(51) Int. Cl. A61M 25/00; A61M 25/01

(52) U.S. Cl. CPC A61M 25/0021; A61M 25/0108; A61B 2017/22079

(57) ABSTRACT

A neurovascular access catheter can comprise an elongate, flexible tubular body. The tubular body can comprise a proximal end, a distal end, and a side wall at least partially defining a central lumen. The central lumen can extend axially through the side wall. The tubular body can include a distal microcatheter segment that extends proximally from the distal end. The tubular body can include a proximal shaft that extends distally from the proximal end. The tubular body can include a tapered dilator segment being positioned in between the distal microcatheter segment and the proximal shaft segment.

### CROSS-REFERENCE TO RELATED APPLICATIONS

This application is a continuation-in-part application of U.S. patent application Ser. No. 17/822,933, filed Aug. 29, 2022, which claims the priority benefit of U.S. Provisional Patent Application Ser. No. 63/239,256, filed Aug. 31, 2021, the contents of each of which are herein incorporated by reference in their entirety.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

In the context of ischemic stroke, a wide variety of thrombectomy devices have been developed to capture and retrieve a clot. These include catheters or wires which carry any of a variety of expandable cages, baskets, snares, drug or energy delivery and aspiration with or without mechanical disruption. Each of these catheters may be called upon to navigate deep into the vasculature, such as distal to the ophthalmic artery. Navigational challenges may limit the ability for many catheters to successfully reach the obstruction. Proximal retraction of the catheter may also result in tip detachment such as when the marker band engages an obstruction.

Notwithstanding the foregoing, there remains a need for new devices and methods for treating vasculature occlusions in the body, including acute ischemic stroke and occlusive cerebrovascular disease, with improved navigational abilities to traverse tortuous vasculature and reach remote treatment sites, and/or improved tensile strength to reduce the risk of tip detachment.

### SUMMARY

One aspect of the disclosure comprises a neurovascular access catheter having a large diameter proximal obturator section and a small diameter distal microcatheter section. The catheter comprises an elongate, flexible tubular body having a proximal end, a distal end, and a side wall at least partially defining a central lumen, the central lumen extending axially therethrough. A distal microcatheter segment extends proximally from the distal end, the distal microcatheter segment comprising a length of about 2 cm to about 10 cm, and an outer diameter of no more than about 0.89 cm (0.035 inches). A proximal shaft segment extends distally from a hub on the proximal end. The proximal shaft segment comprises a length of at least about 140 cm; and an outer diameter of at least about 0.1524 cm (0.06 inches). A tapered dilator segment is positioned in between the distal microcatheter segment and the proximal shaft segment. The neurovascular access catheter may further comprise a coil in the side wall, the coil extending proximally through the distal microcatheter segment and at least partially through the tapered dilator segment. A tubular braid may be provided in the side wall, the tubular braid extending distally from the proximal end. At least a portion of the tubular braid may extend over a proximal end of the coil.

The neurovascular access catheter may further comprise a radiopaque marker at the distal end. At least a distal end of the tapered dilator segment may be visible under fluoroscopy. The tapered dilator segment may comprise a radiopaque dopant, and the dopant may be dispersed throughout the tapered dilator segment. A radiopaque marker may be positioned adjacent a transition between the tapered dilator segment and the distal microcatheter segment.

A wall thickness of the distal microcatheter segment may be no more than about 0.01778 cm (0.007 inches), and a wall thickness of the proximal shaft segment may be at least about 0.071 cm (0.028 inches). A length of the tapered dilator segment of the tubular body between the distal microcatheter segment and the proximal shaft segment may be between about 3 cm and about 6 cm. The distal microcatheter segment may include a distal section comprising an outside diameter of no more than about 0.1016 cm (0.040 inches); and a proximal section comprising an outside diameter of no more than about 0.1778 cm (0.070 inches).

There is also provided a method of treating a patient. The method comprises advancing a distal microcatheter segment of an access catheter across a clot; and advancing an aspiration catheter over the access catheter; wherein an inner diameter of the aspiration catheter is no more than about 0.0254 cm (0.01 inches) larger than an outer diameter of the access catheter. The method may further comprise advancing an intravascular device through the access catheter. The intravascular device may comprise a stent retriever.

There is also provided an access system for navigating an aspiration catheter beyond the ophthalmic artery and for crossing a clot to provide access for a stent retriever. The access system comprises an access catheter having a microcatheter distal section having a first diameter and an obturator proximal section, having a second, larger diameter. The system also includes an aspiration catheter advanceable over the access catheter. A sliding fit between a distal end of the aspiration catheter and the obturator proximal section of the access catheter minimizes a ledge at the distal end of the aspiration catheter to minimize engagement with the ostium to the ophthalmic artery. The microcatheter distal section may comprise an outside diameter of no more than about 0.102 cm (0.040 inches). The aspiration catheter may comprise an outside diameter of at least about 0.178 cm (0.070 inches).

There is also provided a method of treating a patient. The method comprises the steps of providing an access catheter, the access catheter having a distal microcatheter section comprising an outside diameter of no more than about 0.102 cm (0.040 inches) and a length of about 2 cm to about 10 cm, and a proximal shaft comprising an outside diameter of at least about 0.15 cm (0.06 inches); and advancing the distal microcatheter section across a clot. The method may further comprise advancing an aspiration catheter over the access catheter, and may alternatively or in addition comprising advancing an intravascular device through the access catheter. The intravascular device may be a stent retriever.

In some embodiments, a neurovascular catheter may have a pre shaped distal tip for self-orienting with the natural curvature of a vessel to improve transvascular navigation through tortuous distal vasculature. The catheter comprises an elongate flexible tubular body, having a proximal end, an inclined distal end and a side wall defining a central lumen. A distal leading tip is carried on a first side of the inclined distal end, and a preset curve is provided in a distal zone of the tubular body. The distal leading tip lies on a concave side of the curve.

A tubular radiopaque marker may be embedded in the side wall, the tubular radiopaque marker comprising a proximal face and a distal face, wherein the distal face of the radiopaque marker inclines at an angle within a range of from about 45 degrees to about 80 degrees relative to the longitudinal axis of the central lumen.

The central lumen terminates distally in a distal port having an elliptical opening, and the elliptical opening may have an area that is at least about 105% or at least about 110% and generally within the range of from about 110% to about 125% of the cross-sectional area of the central lumen.

The elliptical opening defines an inclined distal face that inclines at an angle within a range of from about 55 degrees to about 65 degrees relative to the longitudinal axis of the central lumen.

The distal face of the radiopaque marker may also incline at an angle within the range of from about 55 degrees to about 75 degrees relative to the longitudinal axis of the central lumen. The proximal face on the radiopaque marker may be approximately perpendicular to the longitudinal axis.

The distal end of the catheter may be spaced apart from the distal face of the radiopaque marker to form an advance segment of the tubular body. The advance segment may have an axial length within a range of from about 0.1 mm to about 5 mm. The axial length of the advance segment on a leading-edge side of the tubular body may be greater than the axial length of the advance segment on a trailing edge side of the tubular body. The axial length of the advance segment on the leading-edge side of the tubular body may be at least about 20% longer than the axial length of the advance segment on the trailing edge side of the tubular body.

The radiopaque marker may have at least one axial slit.

The catheter may further comprise a support filament for increasing the tension resistance and/or influencing the bending characteristics in the distal zone. The support filament may comprise an axially extending filament, which may be carried between an inner liner and the helical coil, and may be positioned on the convex side of the preset curve. In one implementation, the axially extending filament may comprise Vectran™.

In some embodiments, there is provided a self-orienting catheter. The catheter comprises an elongate flexible tubular body, having a proximal end, a distal zone and a side wall defining a central lumen. A tubular radiopaque marker band may be embedded in the side wall in the distal zone. The radiopaque marker band may have a first axial length measured along the side wall at a first circumferential position, and a second, longer axial length measured along the side wall at a second circumferential position offset around the circumference of the catheter by about 180 degrees from the first position; and the tubular body may have a preset curve in the distal zone. The preset curve has a concave side and a convex side, and the second, longer axial length side of the marker may be on the concave side of the curve. An axially extending filament may be positioned on the convex side.

There is also provided a catheter such as a neurovascular catheter with enhanced tensile strength, comprising an elongate flexible tubular body, having a proximal end, a distal end, and a side wall defining a central lumen; a radiopaque marker adjacent the distal end, extending at least part way around a circumference of the tubular body; and a tensile support extending axially in the side wall. The tensile support is attached to the marker to tether the marker to the catheter body to resist distal tip detachment during proximal retraction past an obstruction. In one implementation, the tensile support may extend distally along a first (e.g., inside) side of the radiopaque marker, fold around a distal edge of the radiopaque marker, and extends along a second (e.g., outside) side of the radiopaque marker.

The tensile support may comprise a plurality of fibers and in one example comprises Vectran' multifilament liquid crystal polymer fiber. The tensile support may extend circumferentially at least about 180 degrees or 360 degrees or more around the marker. The sidewall of the catheter may comprise an inner liner, a tie layer and a helical coil, and the tensile support extends axially between the helical coil and the inner liner. The side wall may include an outer jacket comprising a plurality of tubular segments, a proximal tubular segment of the plurality of tubular segments having a durometer of at least about 60 D, and a distal tubular segment of the plurality of tubular segments having a durometer of at most about 35 D.

The radiopaque marker may comprise a proximal face and a distal face, and the distal face may incline at an angle within a range of from about 45 degrees to about 80 degrees relative to the longitudinal axis of the central lumen. The radiopaque marker may comprise an annular ring with at least one axial slit.

The catheter may comprise an inclined distal face with a distal port having an elliptical opening, and the elliptical opening may comprise an area that is at least about 105% of a transverse cross-sectional area of the central lumen. The area of the elliptical opening may be at least about 110% of the cross-sectional area of the central lumen, and the elliptical opening may lie on a plane that inclines at an angle within a range of from about 55 degrees to about 65 degrees relative to the longitudinal axis of the central lumen.

A proximal face on the radiopaque marker may be approximately perpendicular to the longitudinal axis. The distal end of the catheter may be spaced apart from the distal face of the radiopaque marker to form an advance segment of the tubular body beyond the distal end of the marker. The advance segment may have an axial length within a range of from about 0.1 mm to about 5 mm. The axial length of the advance segment on a leading-edge side of the tubular body may be greater than the axial length of the advance segment on a trailing edge side of the tubular body.

The catheter may be configured to withstand at least about 0.68 kg (1.5 pounds) or at least about 1.59 kg (3.5 pounds) tension before failure (tip detachment) and in some implementations at least about 2.27 kg (5 pounds) tension before failure, or at least about 3.18 kg (7 pounds) tension before failure, in a catheter having an outside diameter of no more than about 0.25 cm (0.10 inches) or no more than about 0.20 cm (0.080 inches).

In any of the neurovascular catheters described herein, the radiopaque marker may comprise a tubular side wall having a proximal end and a distal end, and at least one compression feature to increase the compressibility of the proximal end. The compression feature may comprise at least one compression gap in the side wall, opening at the proximal end of the sidewall and extending in a distal direction. Alternatively, the compression feature may comprise a plurality of struts joined at apexes to form a collapsible tubular side wall attached to the coil or other catheter component.

### BRIEF DESCRIPTION OF THE DRAWINGS

FIG. 1A is a side elevational view of an embodiment of a catheter.

FIG. 1B is a side elevational view of an embodiment of a catheter with a preshaped curve.

FIG. 1C is an enlargement of a distal section of the catheter of FIG. 1.

FIG. 1D is another enlargement of a distal section of the catheter of FIG. 1.

FIG. 2 illustrates a cross-sectional elevational view of a catheter wall according to another embodiment.

FIG. 3A illustrates a cross-sectional elevational view of a catheter wall according to another embodiment, showing one or more axially extending tension elements.

FIG. 3B describes a side elevational view of the catheter of FIG. 3A

FIG. 3C illustrates a cross-sectional view taken along the line C-C of FIG. 3B, showing one or more axially extending tension elements.

FIG. 3D is a side elevational cross section through an angled distal catheter or extension tube tip.

FIG. 3E illustrates a tip as in FIG. 3D, with a tethered marker band.

FIGS. 4A-4B are side elevational views of marker bands.

FIG. 4C is a top plan view of the marker band of FIG. 4B.

FIG. 4D is a side elevational view of an alternative marker band.

FIG. 4E is a side elevational view of an alternative marker band with an integral tubular tension element.

FIG. 4F is a side elevational view of an alternative marker band.

FIG. 4G is a side perspective view of the alternative marker band of FIG.

4F.

FIG. 4H is a side elevational view of an alternative marker band.

FIG. 4I is a side perspective view of the alternative marker band of FIG.

FIG. 5A illustrates a side elevational view of a progressively enhanced flexibility catheter according to an embodiment.

FIG. 5B is a proximal end view of the enhanced flexibility catheter of FIG. 5A.

FIGS. 6A-6C illustrates various methods of utilizing various access catheters.

FIG. 7 illustrates a side view of an example access catheter.

FIG. 8 illustrates a side cross-sectional view of the example access catheter of FIG. 7.

FIG. 9 illustrates a side view of an example access catheter.

FIG. 10 illustrates a side cross-sectional view of the example access catheter of FIG. 9.

FIG. 11 illustrates a side view of an example access catheter.

FIG. 12 illustrates a side cross-sectional view of the example access catheter of FIG. 11.

FIG. 13 illustrates an example method of using an access catheter to remove a clot.

FIG. 14 illustrates an example method of using an access catheter to remove a clot.

FIG. 15 illustrates an example system for accessing a vessel.

FIG. 16 illustrates an example system for accessing a vessel.

FIG. 17 illustrates an example system for accessing a vessel.

### DETAILED DESCRIPTION

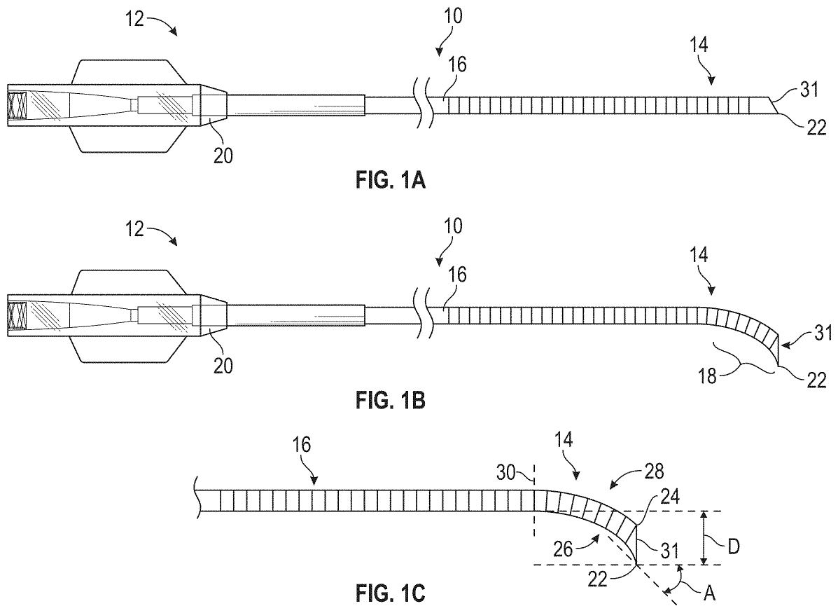

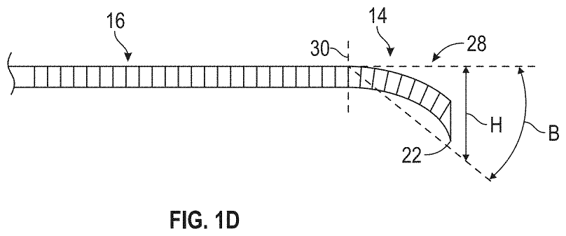

Referring to FIG. 1A-1C, there is disclosed a catheter 10. Although primarily described in the context of an aspiration catheter with a single central lumen, catheters can readily be modified to incorporate additional structures, such as permanent or removable column strength enhancing mandrels, two or more lumen such as to permit drug, contrast or irrigant infusion or to supply inflation media to an inflatable balloon carried by the catheter, or combinations of these features, as will be readily apparent to one of skill in the art in view of the disclosure herein. In addition, the disclosure will be described primarily in the context of removing obstructive material from remote vasculature in the brain, but has applicability as an access catheter for delivery and removal of any of a variety of diagnostics or therapeutic devices with or without aspiration and/or within any suitable vessel within a patient.

The catheters disclosed herein may readily be adapted for use throughout the body wherever it may be desirable to distally advance a low-profile high flexibility catheter into small and/or tortuous vasculature. For example, catheter shafts may be dimensioned for use throughout the coronary and peripheral vasculature, the gastrointestinal tract, the urethra, ureters, Fallopian tubes and other lumens and potential lumens, as well. The catheter shaft construction may also be used to provide minimally invasive percutaneous tissue access, such as for diagnostic or therapeutic access to a solid tissue target (e.g., breast or liver or brain biopsy or tissue excision), delivery of laparoscopic tools or access to bones such as the spine for delivery of screws, bone cement or other tools or implants.

The catheter 10 generally comprises an elongate tubular body 16 extending between a proximal end 12 and a distal functional end 14. The catheter 10 may have no preset curve (FIG. 1A) or may have a preset curve (FIGS. 1B-1C). The length of the tubular body 16 depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site, as discussed in further detail below.

Catheters may have a length and diameter suitable for the intended access point and target location. In one example, referring to FIGS. 1A-1C, the catheter 10 may have an effective length from the manifold or hub 20 to distal tip 22 generally no more than about 180 cm or no more than about 160 cm. and typically from about 70 cm to about 150 cm, from about 90 cm to about 130 cm, or from about 105 cm to about 115 cm. The outer diameter of the catheter 10 may be from about 0.089 cm (0.035 inches) to about 0.381 cm (0.15 inches), from about 0.23 cm (0.09 inches) to about 0.33 cm (0.13 inches), and may be lower in a distal segment than in a proximal segment.

The inner diameter of the catheter 10 in a single central lumen embodiment may be greater than or equal to about 0.254 cm (0.1 inches), greater than or equal to about 0.22 cm (0.088 inches), or greater than or equal to about 0.20 cm (0.08 inches), or greater than or equal to about 0.15 cm (0.06 inches). The inner diameter of the catheter 10 in a single central lumen embodiment may be less than about 0.51 cm (0.20 inches) or 0.381 cm (0.15 inches), or less than or equal to about 0.28 cm (0.11 inches), less than or equal to about 0.25 cm (0.1 inches), less than or equal to about 0.22 cm (0.088 inches), or less than or equal to about 0.18 cm (0.07 inches), and often no more than about 0.24 (0.095 inches).

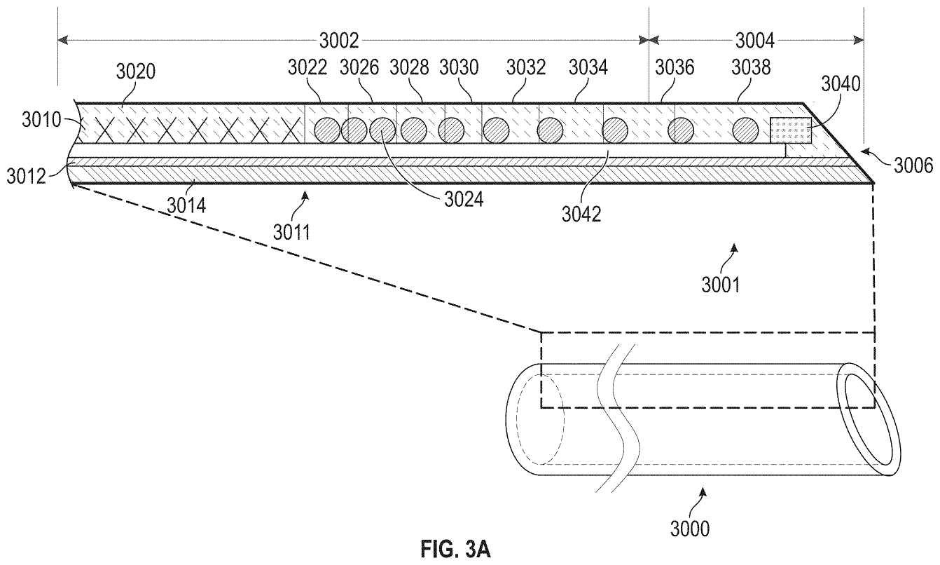

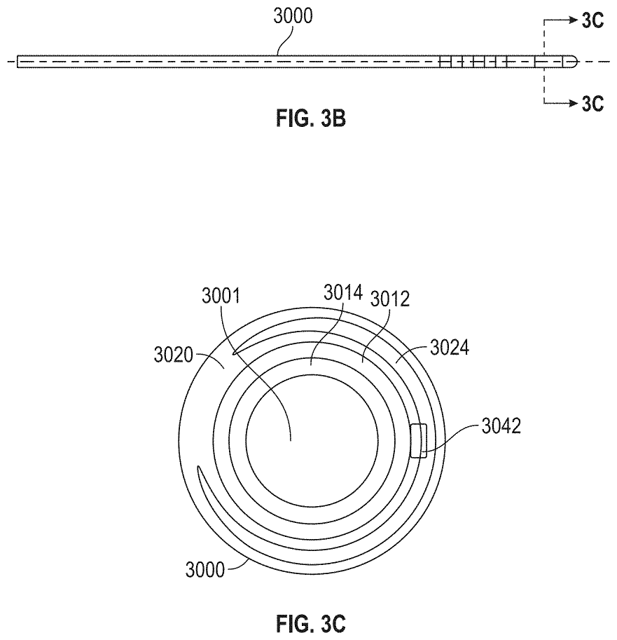

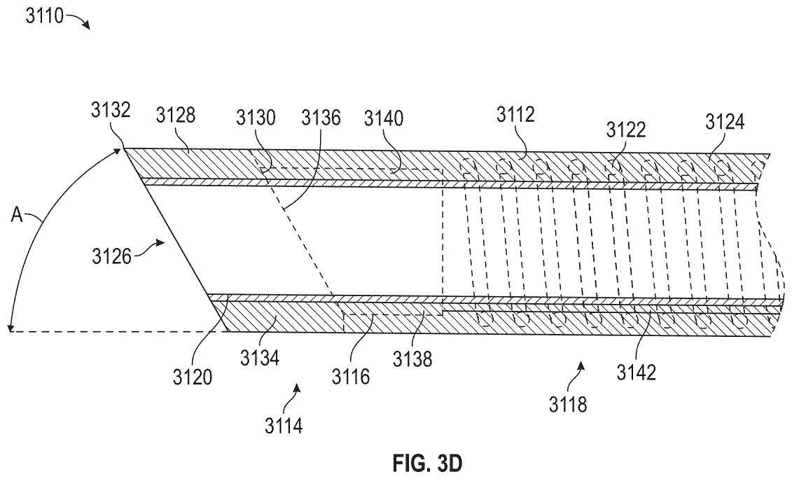

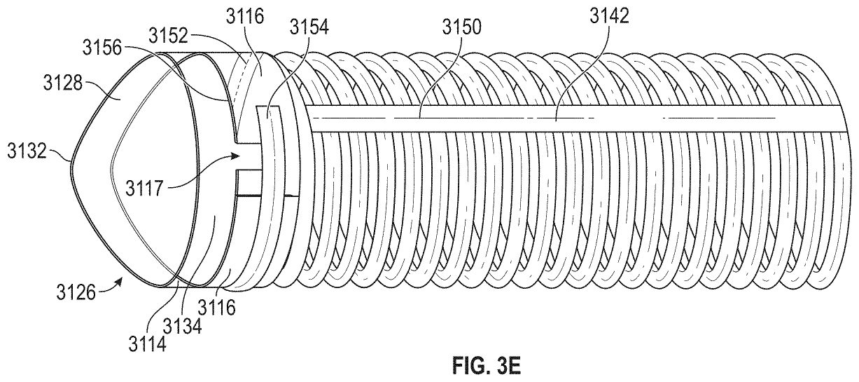

FIG. 1C illustrates a distal section of the tubular body 16. A passive distal steering zone 18 on the tubular body 16 is provided with a pre-shaped curve, having a concave side 26 and a convex side 28. The tubular body 16 is additionally provided with an inclined face 31, discussed in greater detail in connection with FIG. 3D. The inclined face 31 produces a leading edge at distal tip 22, and an opposing trailing edge 24. The leading edge 22 is disposed on the concave side 26 of the pre-shaped curve.

In an unconstrained configuration, the pre-shaped curve establishes an angle A between the longitudinal axis of the tubular body 16 proximally of the curve (i.e., concave side of the pre-shaped curve), and the longitudinal axis of the distal most 2 or 3 mm of the tubular body 16. Angle A is generally within the range of from about 25° to about 55°, preferably no more than about 50°, and in some implementations between about 30° and about 40°. The angle A is preferably within the range of from about 32° and about 38°, and in one example is about 35°. Additionally, in some implementations, angle A is within the range of from about 25° to about 45°, about 20° to about 45°, about 15° to about 55°, or about 15° to about 50°. The angle is low enough that in combination with the low lateral bias of the preset curve, the tip 22 of the catheter will follow the native vasculature but not penetrate the side wall into extravascular space.

In some embodiments, as shown in FIG. 1D, the pre-shaped curve establishes an angle B between the longitudinal axis of the tubular body 16 proximally of the curve (e.g., at transition 30 or at convex side of the pre-shaped curve or based on proximal edge of markerband), and the distal tip 22. Angle B is generally within the range of from about 25° to about 55°, preferably no more than about 50°, and in some implementations between about 25° and about 35°. The angle A is preferably within the range of from about 27° and about 33°, and in one example is about 30°. Additionally, in some implementations, angle B is within the range of from about 25° to about 45°, about 20° to about 45°, about 15° to about 55°, or about 15° to about 50°.

In some embodiments, as further shown in FIG. 1D, the pre-shaped curve establishes a height H between the longitudinal axis of the tubular body 16 proximally of the curve (e.g., at transition 30 or at convex side of the pre-shaped curve or based on proximal edge of markerband), and the distal tip 22. Height H is generally within the range of from about 0.25 cm to about 0.6 cm, and in some implementations, between about 0.3 cm to about 0.4 cm or about 0.35 cm to about 0.45 cm or about 0.4 cm to about 0.5 cm or about 0.45 cm to about 0.55 cm.

The lateral limit of unconstrained deflection D is generally within the range of from about 0.3 cm (0.1 inches) and about 0.5 cm (0.2 inches) and, in one embodiment, is about 0.38 cm (0.15 inches). In some implementations, unconstrained deflection D is generally within the range of from about 0.13 cm (0.05 inches) to about 0.38 cm (0.15 inches), about 0.15 cm (0.06 inches) to about 0.33 cm (0.13 inches), about 0.18 cm (0.07 inches) to about 0.31 cm (0.12 inches), about 0.191 cm (0.075 inches) to about 0.31 cm (0.12 inches), etc. Unconstrained deflection D will typically be no greater than about 0.8 cm (0.3 inches) or about 0.64 cm (0.25 inches) or about 0.5 cm (0.2 inches), depending upon the catheter diameter.

The tubular body 16 includes a transition 30 at the proximal limit of the pre-shaped curve. The arc length of the pre shaped curve measured from the transition 30 to the distal tip 22 is generally less than about 2 cm or less than about 1.5 cm or less than about 1 cm, and often between about 0.5 cm and about 1.5 cm and in some implementations the length is about 1.1 cm to about 1.4 cm. In some implementations, the arc length is within the range of from about 0.75 cm to about 1.75 cm or about 0.5 cm to about 2 cm or about 1.25 cm to about

The tubular body 16 can be sufficiently flexible that the catheter is trackable for ease of advancement through even narrow and/or tortuous body passageways. The catheter may bend in any plane in three-dimensional space, in response to advancing through the curvature of the vasculature. Thus, at least the distal preshaped curve may spontaneously twist about the longitudinal axis of the catheter during advancement through a body passageway as itself orients to follow the lowest energy state configuration through the curvature of the vessel. Torque transmission through the tubular body 16 is sufficiently low (low torsional stiffness) that the distal end of the catheter is able to twist about its axis as desired in both clockwise and counterclockwise directions to self-orient with the vasculature during distal advance, without needing the proximal end of the catheter to rotate. In some embodiments, the distal end of the catheter can twist at least about 10 degrees, at least about 20 degrees or in some implementations at least about 45 degrees or 90 degrees or more in either direction without any rotation of the proximal end of the catheter. The self-orientation or twisting of the catheter may optimize an angle of interaction of the distal end of the catheter with a clot to improve or maximize clot ingestion.

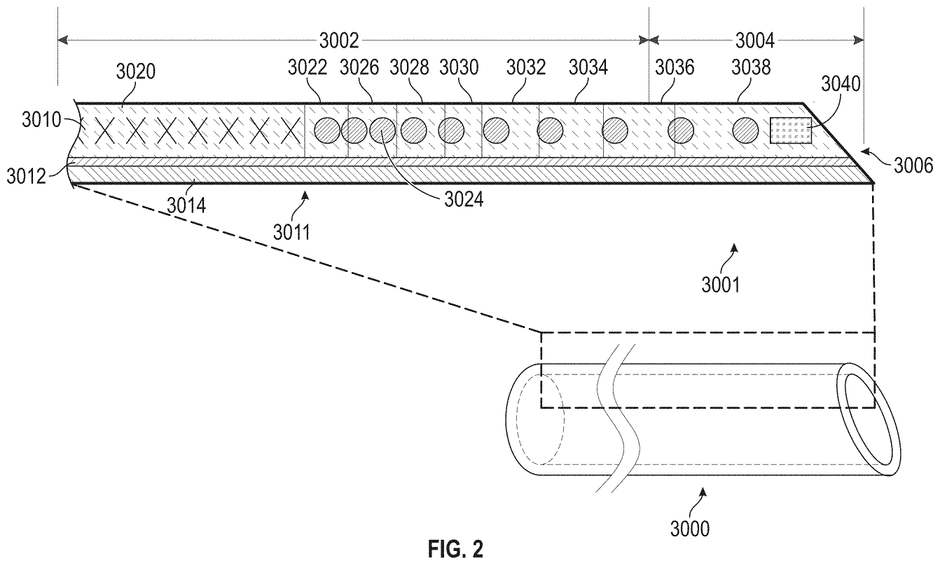

FIG. 2 illustrates a cross section through the sidewall of a distal portion of a single lumen catheter that may be formed either with or without the preset curve. Adjacent loops or filars of the coil 3024 may have a constant pitch throughout the length of the coil or may be closely tightly wound in a proximal zone with a distal section having looser spacing between adjacent loops. In an embodiment having a coil section 3024 with an axial length of at least between about 20% and about 30% of the overall catheter length, (e.g., 28 cm coil length in a 110 cm catheter shaft 16), at least the distal about 1 cm or about 2 cm or about 3 cm or about 4 cm of the coil will have a spacing that is at least about 130%, and in some implementations at least about 150% or more than the spacing in the proximal coil section. In a 110 cm catheter shaft 3000 having a Nitinol coil, the spacing in the proximal coil may be about 0.010 cm (0.004 inches) and in the distal section may be at least about 0.015 cm (0.006 inches) or about 0.018 cm (0.007 inches) or more.

The distal end of the coil 3024 can be spaced proximally from the distal end of the inner liner 3014, for example, to provide room for an annular radiopaque marker 3040. The coil 3024 may be set back proximally from the distal end, in some embodiments, by approximately no more than about 1 cm, about 2 cm, or about 3 cm. In one embodiment, the distal end of the catheter 10 is provided with a beveled distal surface 3006 residing on a plane having an angle of at least about 10 degrees or about 20 degrees and in one embodiment about 30 degrees with respect to a longitudinal axis of the catheter 10. The radiopaque marker 3040 may reside in a plane that is transverse to the longitudinal axis. Alternatively, at least the distally facing edge of the annular radiopaque marker 3040 may be an ellipse, residing on a plane which is inclined with respect to the longitudinal axis to complement the bevel angle of the distal surface 3006. Additional details are described in connection with FIG. 3D below.

After applying the proximal braid 3010 over tie layer 3012, the distal coil 3024 and the RO marker 3040 are provided with an outer jacket 3020 such as a shrink wrap tube to enclose the catheter body 16. The outer shrink-wrapped sleeve 3020 may comprise any of a variety of materials, such as polyethylene, polyurethane, polyether block amide (e.g., PEBAX®), nylon or others known in the art. Sufficient heat is applied to cause the polymer to flow into and embed the proximal braid and distal coil.

In one implementation, the outer shrink wrap jacket 3020 is formed by sequentially advancing a plurality of short tubular segments 3022, 3026, 3028, 3030, 3032, 3034, 3036, 3038 concentrically over the catheter shaft subassembly, and applying heat to shrink the sections on to the catheter 10 and provide a smooth continuous outer tubular body. The foregoing segmented construction may extend along at least the most distal about 10 cm, and preferably at least about the most distal about 20 cm, about 25 cm, about 30 cm, about 35 cm, about 40 cm, or more than about 40 cm of the catheter body 10. The entire length of the outer shrink wrap jacket 3020 may be formed from tubular segments and the length of the distal tubular segments (e.g., 3022, 3026, 3028, 3030, 3032, 3034, 3036, 3038) may be shorter than the one or more tubular segments forming the proximal portion of the outer shrink wrap jacket 3020 in order to provide proximal backup support and steeper transitions in flexibility toward the distal end of the catheter 10.

The durometer of the outer wall segments may decrease in a distal direction. For example, proximal segments such as 3022 and 3026, may have a durometer of at least about 60 D or about 70 D, with gradual decrease in durometer of successive segments in a distal direction to a durometer of no more than about 35 D or about 25 D or lower. A 25 cm section may have at least about 3 or about 5 or about 7 or more segments and the catheter 10 overall may have at least about 6 or about 8 or about 10 or more distinct flexibility zones. The distal 1 or 2 or 4 or more segments 3036, 3038, may have a smaller OD following shrinking than the more proximal segments 3022-3034 to produce a step down in OD for the finished catheter body 16. The length of the lower OD section 3004 may be within the range of from about 3 cm to about 15 cm and, in some embodiments, is within the range of from about 5 cm to about 10 cm such as about 7 cm or about 8 cm, and may be accomplished by providing the distal segments 3036, 3038 with a lower wall thickness.

In another embodiment, the most distal portion of the catheter 10 may comprise a durometer of less than approximately 35 D (e.g., 25 D) to form a highly flexible distal portion of the catheter and have a length between approximately 25 cm and approximately 35 cm. The distal portion may comprise one or more tubular segments of the same durometer (e.g., segment 3038). A series of proximally adjacent tubular segments may form a transition region between a proximal stiffer portion of the catheter 3000 and the distal highly flexible portion of the catheter. The series of tubular segments forming the transition region may have the same or substantially similar lengths, such as approximately 1 cm.

The relatively short length of each of the series of tubular segments may provide a steep drop in durometer over the transition region. For example, the transition region may have a proximal tubular segment 3036 (proximally adjacent the distal portion) having a durometer of approximately 35 D. An adjacent proximal segment 3034 may have a durometer of approximately 55 D. An adjacent proximal segment 3032 may have a durometer of approximately 63 D. An adjacent proximal segment 3030 may have a durometer of approximately 72 D.

More proximal segments may comprise a durometer or durometers greater than approximately 72 D and may extend to the proximal end of the catheter or extension catheter segment. For instance, an extension catheter segment may comprise a proximal portion greater than approximately 72 D between about 1 cm and about 3 cm. In some embodiments, the proximal portion may be about 2 cm long. In some embodiments, the most distal segments (e.g., 3038-3030) may comprise PEBAX® or Neusoft™, and more proximal segments may comprise a generally stiffer material, such as Vestamid® or, Grilamid®.

The inner diameter of the catheter 10 may be between approximately 0.15 cm (0.06 inches) and 0.20 cm (0.08 inches), between approximately 0.165 cm (0.065 inches) and 0.190 cm (0.075 inches), or between approximately 0.173 cm (0.068 inches) and 0.185 cm (0.073 inches). In some embodiments, the inner diameter is approximately 0.180 cm (0.071 inches).

In some embodiments, the distal most portion may taper to a decreased inner diameter as described elsewhere herein. The taper may occur approximately between the distal highly flexible portion and the transition region (e.g., over the most proximal portion of the distal highly flexible portion). The taper may be relatively gradual (e.g., occurring over approximately 10 or more cm) or may be relatively steep (e.g., occurring over less than approximately 5 cm). The inner diameter may taper to an inner diameter between about 0.05 cm (0.02 inches) and about 0.15 cm (0.06 inches). For example, the inner diameter may be about 0.066 cm (0.026 inches), about 0.089 cm (0.035 inches), about 0.114 cm (0.045 inches), or about 0.140 cm (0.055 inches) at the distal end of the catheter 3000. In some embodiments, the inner diameter may remain constant, at least over the catheter extension segment. In some embodiments, a consideration for inner diameter dimension may be the outer diameter of the guidewire and the clearance needed for the guidewire to travel within the catheter 10. Additionally, another inner diameter consideration may be for the use of internally housed devices, such as, stent retrievers or any other device known in the art requiring a specific housing diameter for proper deployment.

In some embodiments, the coil 3024 may extend proximally from a distal end of the catheter 10 along the highly flexible distal portion ending at the distal end of the transition region. In other embodiments, the coil 3024 may extend from a distal end of the catheter to the proximal end of the transition region, to a point along the transition region, or proximally beyond the transition region. In other embodiments, the coil 3024 may extend the entire length of the catheter 10 or catheter extension segment as described elsewhere herein. The braid 3010, when present, may extend from the proximal end of the coil 3024 to the proximal end of the catheter 10.

Referring to FIGS. 3A-3D, the catheter may further comprise an axial tension element or support such as a ribbon or one or more filaments or fibers for increasing the tension resistance and/or influencing the bending characteristics in the distal zone. The tension support may comprise one or more axially extending mono strand or multi strand filaments 3042. The one or more tension element 3042 may be axially placed inside the catheter wall near the distal end of the catheter. The filament may be positioned on the convex side of a catheter having the preset curve. The one or more tension element 3042 may serve as a tension support and resist elongation of the catheter wall under tension (e.g., when the catheter is being proximally retracted through tortuous or narrowed vasculature).

At least one of the one or more tension element 3042 may proximally extend along the length of the catheter wall from within about 1.0 cm from the distal end of the catheter to less than about 10 cm from the distal end of the catheter, less than about 20 cm from the distal end of the catheter, less than about 30 cm from the distal end of the catheter, less than about 40 cm from the distal end of the catheter, or less than about 50 cm from the distal end of the catheter.

The one or more tension element 3042 may have a length greater than or equal to about 40 cm, greater than or equal to about 30 cm, greater than or equal to about 20 cm, greater than or equal to about 10 cm, or greater than or equal to about 5 cm.

At least one of the one or more tension element 3042 may extend at least about the most distal 50 cm of the length of the catheter, at least about the most distal 40 cm of the length of the catheter, at least about the most distal 30 cm or about 20 cm or about 10 cm of the length of the catheter.

In some implementations, the tension element extends proximally from the distal end of the catheter along the length of the coil 24 and ends proximally within about 5 cm or about 2 cm or less either side of the transition 3011 between the coil 3024 and the braid 3010. The tension element may end at the transition 3011, without overlapping with the braid 3010.

The one or more tension element 3042 may be placed near or radially outside the tie layer 3012 or the inner liner 3014. The one or more tension element 3042 may be placed near or radially inside the braid 3010 and/or the coil 3024. The one or more tension element 3042 may be carried between the inner liner 3014 and the helical coil 3024, and may be secured to the inner liner or other underlying surface by an adhesive prior to addition of the next outer adjacent layer such as the coil.

When more than one tension element 3042 or filament bundles are spaced circumferentially apart in the catheter wall, the tension elements 3042 may be placed in a radially symmetrical manner. For example, the angle between two tension elements 3042 with respect to the radial center of the catheter may be about 180 degrees. Alternatively, depending on desired clinical performances (e.g., flexibility, trackability), the tension elements 3042 may be placed in a radially asymmetrical manner. The angle between any two tension elements 3042 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 3042 may comprise materials such as Vectran™, Kevlar®, Polyester, Meta-Para-Aramide, or any combinations thereof. At least one of the one or more tension element 3042 may comprise a single fiber or a multi-fiber bundle, and the fiber or bundle may have a round or rectangular (e.g., ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 3042, as measured in the radial direction, may be no more than about 2%, 5%, 8%, 15%, or 20% of that of the catheter 10.

The cross-sectional dimension of the one or more tension element 3042, as measured in the radial direction, may be no more than about 0.003 cm (0.001 inches), no more than about 0.005 cm (0.002 inches), no more than about 0.010 cm (0.004 inches), no more than about 0.015 cm (0.006 inches), no more than about 0.020 cm (0.008 inches), or about 0.038 cm (0.015 inches).

The one or more tension element 3042 may increase the tensile strength of the distal zone of the catheter before failure under tension to at least about 0.45 kg (1 pound), at least about 0.90 kg (2 pounds), at least about 1.4 kg (3 pounds), at least about 1.8 kg (4 pounds), at least about 2.3 kg (5 pounds), at least about 2.7 kg (6 pounds), at least about 3.2 kg (7 pounds), at least about 3.6 kg (8 pounds), or at least about 4.5 kg (10 pounds) or more.

Any of the catheters disclosed herein, whether or not an axial tension element is included, may be provided with an angled distal tip. Referring to FIG. 3D, distal catheter tip 3110 comprises a tubular body 3112 which includes an advance segment 3114, a marker band 3116 and a proximal segment 3118. An inner tubular liner 3120 may extend throughout the length of the distal catheter tip 3110, and may comprise dip coated PTFE.

A reinforcing element 3122 such as a braid or spring coil is embedded in an outer jacket 3124 which may extend the entire length of the distal catheter tip 3110.

The advance segment 3114 terminates distally in an angled face 3126, to provide a leading side wall portion 3128 having a length measured between the distal end 3130 of the marker band 3116 and a distal tip 3132. A trailing side wall portion 3134 of the advance segment 3114, has an axial length in the illustrated embodiment of approximately equal to the axial length of the leading side wall portion 3128 as measured at approximately 180 degrees around the catheter from the leading side wall portion 3128. The leading side wall portion 3128 may have an axial length within the range of from about 0.1 mm to about 5 mm and generally within the range of from about 1 mm to about 3 mm. The trailing side wall portion 3134 may be at least about 0.1 mm or about 0.5 mm or about 1 mm or about 2 mm or more shorter than the axial length of the leading side wall portion 3128, depending upon the desired performance.

The angled face 3126 inclines at an angle A within the range of from about 45 degrees to about 80 degrees from the longitudinal axis of the catheter. For certain implementations, the angle is within the range of from about 55 degrees to about 65 degrees or within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105%, and no more than about 130%, in some implementations within the range of from about 110% and about 125% and in one example is about 115%.

In the illustrated embodiment, the axial length of the advance segment is substantially constant around the circumference of the catheter, so that the angled face 3126 is approximately parallel to the distal surface 3136 of the marker band 3116. The marker band 3116 has a proximal surface approximately transverse to the longitudinal axis of the catheter, producing a marker band 3116 having a right trapezoid configuration in a side elevational view. A short sidewall 3138 is rotationally aligned with the trailing side wall portion 3134, and has an axial length within the range of from about 0.2 mm to about 4 mm, and typically from about 0.5 mm to about 2 mm. An opposing long sidewall 3140 is rotationally aligned with the leading side wall portion 3128. Long sidewall 3140 of the marker band 3116 is generally at least about 10% or 20% longer than short sidewall 3138 and may be at least about 50% or 70% or 90% or more longer than short sidewall 3138, depending upon desired performance. Generally, the long sidewall 3140 will have a length of at least about 0.5 mm or 1 mm and less than about 5 mm or about 4 mm.

Any of the marker bands described herein may be a continuous annular structure, or may optionally have at least one and optionally two or three or more axially extending slits 3117 throughout its length. The slit may be located on the short sidewall 3138 or the long sidewall 3140 or in between, depending upon desired bending characteristics. Any of the marker bands described herein may comprise any of a variety of radiopaque materials, such as a platinum/iridium alloy, with a wall thickness preferably no more than about 0.008 cm (0.003 inches) and in one implementation is about 0.003 cm (0.001 inches). In one implementation, at least one axial slit is aligned with the convex side of the preset curve, and the filament extends distally beyond the proximal face of the marker and into the axial slit.

The marker band zone of the assembled catheter may have a relatively high bending stiffness and high crush strength, such as at least about 50% or at least about 100% less than proximal segment 18 but generally no more than about 200% less than proximal segment 3118. The high crush strength may provide radial support to the adjacent advance segment 3114 and particularly to the leading side wall portion 3128, to facilitate the functioning of distal tip 3132 as an atraumatic bumper during transluminal advance and to resist collapse under vacuum. The proximal segment 3118 preferably has a lower bending stiffness than the marker band zone, and the advance segment 3114 preferably has even a lower bending stiffness and crush strength than the proximal segment 3118.

The advance segment 3114 may comprise a distal extension of the outer jacket 3124 and optionally the inner liner 3120, without other internal supporting structures distally of the marker band 3116. Outer jacket may comprise extruded Tecothane™. The advance segment 3114 may have a bending stiffness and radial crush stiffness that is no more than about 50%, and in some implementations no more than about 25% or 15% or 5% or less than the corresponding value for the proximal segment 3118.

A tension element 3142 with dimensions and materials as has been discussed elsewhere herein extends through at least a distal portion of the length of the proximal segment 3118. As illustrated, the tension element 3142 may terminate distally at a proximal surface of the marker band 3116 and extend axially radially outwardly of the tubular liner 3120 and radially inwardly from the support coil 3122. Alternatively, the marker band may be provided with at least one or two axially extending slits 3117, and the fiber can extend into the slit, thus axially overlapping with the marker band. Tension element 3142 may extend substantially parallel to the longitudinal axis, or may be inclined into a mild spiral having no more than 10 or 7 or 3 or 1 or less complete revolutions around the catheter along the length of the spiral. The fiber may comprise a high tensile strength material such as a multifilament yarn spun from liquid crystal polymer such as a Vectran™ multifilament LCP fiber.

In the implementation illustrated in FIG. 3E, the tension element 3142 extends axially in a distal direction along the outside of (or inside of) the coil, towards an anchor which may be in the form of a continuous or slit annular ring such as the marker band 3116 which may have an inclined distal face as has been discussed. The tension element 3142 is preferably secured to the anchor, to increase the tensile force threshold before failure by tip detachment. This allows the catheter to be pulled proximally through restrictions such as a vascular restriction or a kink in the guide catheter which may collapse but not detach the marker band. The enhanced tensile strength also provides tactile feedback to the physician when they encounter a restriction that may shear off the marker band. In an implementation having a slit 3117, the axis of the tension element 3142 may be circumferentially offset from the slit 3117 to avoid the fiber pulling through the slit.

The tension element 3142 may be secured to the anchor in any of a variety of ways, depending upon the structures and materials involved, including adhesives, welding or mechanical interference fit. In the illustrated implementation, the tension element 3142 is wrapped around at least a distally facing edge of the marker band 3116, such as the distal edge of the marker band or a proximal edge of an aperture through the marker band 3116. In one implementation, the tension element 3142 extends axially along a first surface of the marker band beyond the marker band and is folded back around the distal edge of the marker band, and onto a second surface of the marker band and secured to the tubular body (e.g., to the marker band, or to itself).

In the illustrated example, a first segment 3150 of the tension element 3142 extends axially along the catheter body over or preferably under the coil, under the marker band 3116 and distally along the inside surface of the marker band to the distal edge 3156 of the marker band. The tension element is folded back over the distal edge 3156 and extends proximally over the outside surface of the marker band along an angled segment 3152 and wrapped in a circumferential direction around the tubular body such as over the marker band 3116 and/or adjacent catheter side wall to an end 3154. The tension element may be wrapped circumferentially around through an angle of at least about 180 degrees and preferably at least about 270 degrees or about 360 degrees or at least about 450 degrees or more. The tension element may be tacked down over the marker band or adjacent catheter shaft with an adhesive such as Loctite® prior to applying the outer polymer jacket.

Alternatively, the tension element may be folded around the marker band and back proximally over itself, running proximally for a bonding zone of at least about 1 or about 2 or about 5 or more cm along which it may be bonded to itself before being encased in the outer jacket.

In the illustrated implementation, the tension element crosses the marker band at a point within the range of approximately 20 degrees to about 40 degrees circumferentially offset from the center of the slit 3117. Alternatively, the tensile element may cross the marker band within the range of approximately 80 degrees to about 100 degrees offset from the slit, or within the range of from about 170 degrees to about 190 degrees from the slit. In an implementation in which the slit is not located at the shortest axial dimension of the marker band, the foregoing offsets may be measured from the shortest axial dimension.

The radial compressibility of the marker band may desirably increase in the proximal direction from the distal end of the marker band to the proximal end of the marker band, to form a continuous or stepped graduated compressibility. This may facilitate radial compressibility at the proximal end of the marker band such as when the marker band encounters an obstruction (e.g., vascular obstruction or kink in the guide catheter) during proximal retraction of the catheter. Further proximal retraction allows the side wall of the marker band to ramp up to the diameter of the distal end of the marker band, displacing the obstruction laterally and/or progressively collapsing the marker band to allow the marker band to squeeze past the obstruction. This, in combination with the attached tensile element, optimizes the likelihood of avoiding marker band detachment.

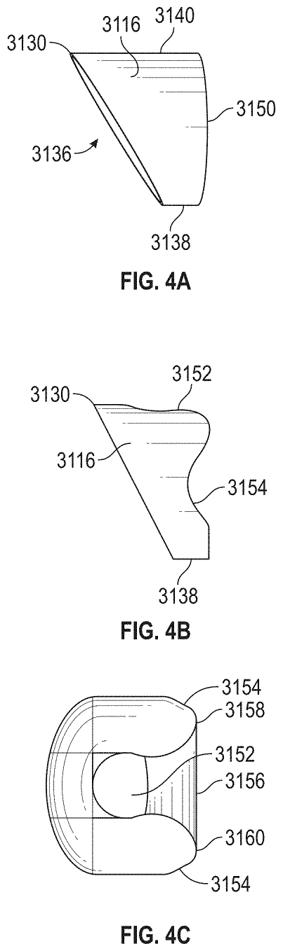

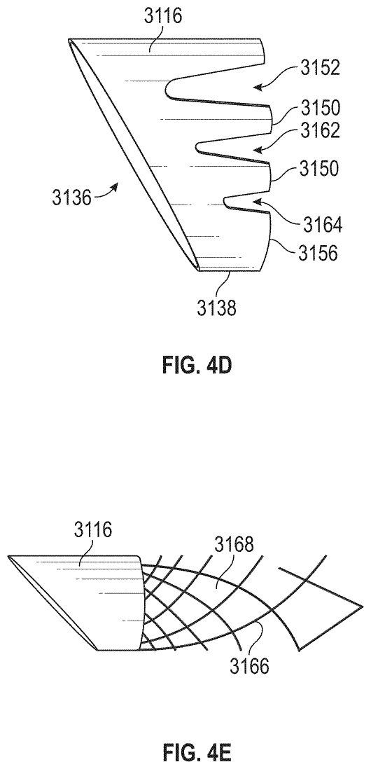

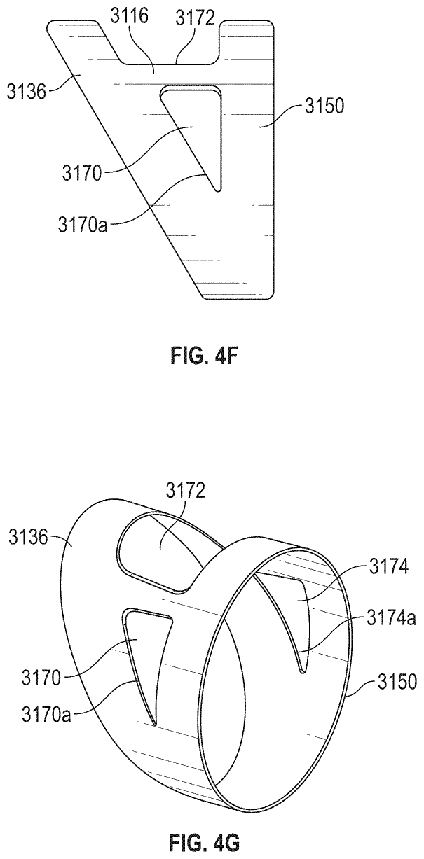

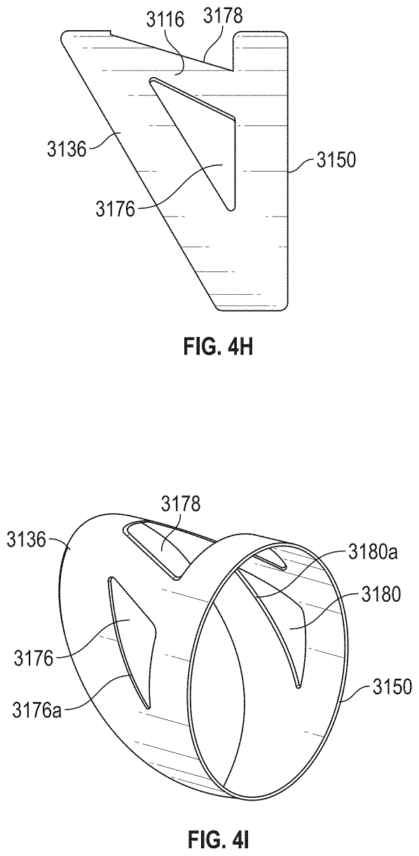

The basic geometry of a previously described marker band 3116 is illustrated in FIG. 4A. Marker band 3116 extends between a proximal transverse face 3150 and a distal inclined face 3136. A long side wall 3140 terminates distally in a distal tip 3130. An opposing short side wall 3138 may contain an axial slit as has been discussed.

Referring to FIG. 4B, a marker band 3116 is provided with a compression feature that increases the radial compressibility of the proximal end of the marker band. In the illustrated implementation, the compression feature comprises at least a first compression gap 3152 and may comprise at least a second compression gap in the form of a proximal facing concavity 3154. The first compression gap 3152 extends distally from the proximal face 3150 at least about 25% and in some implementations at least about 50% or about 70% or more of the length of long sidewall 3140.

The second compression gap may extend distally from the proximal face 3150, rotated approximately 90 degrees in a first circumferential direction from the first compression gap 3152. At least a third compression gap may be provided, rotated about 90 degrees in a second circumferential direction from the first compression gap 3152.

The foregoing construction provides an arcuate base 3156 in the form of the proximal edge of the marker band 3116, lying on the plane of proximal face 3150, for contacting the distal end of the coil or other sidewall reinforcement in the catheter body. A first foot 3158 and a second foot 3160 are also formed, also lying approximately on the plane corresponding to proximal face 3150, for supporting the marker band 3116 against the distal end of the spring coil or other catheter body reinforcement. This allows radial compression of the proximal end of the marker band 3116, while also supporting the marker band 3116 against tilting relative to the distal face of the coil.

In the implementation shown in FIG. 4D, marker band 3116 having the characteristics of the marker band of FIG. 4A is modified by providing at least a first compression gap 3152 that facilitates radial compression. A second compression gap 3162 and optionally a third compression gap 3164 or more may be provided depending upon desired performance. The proximal openings of the compression gaps may reside on a transverse plane, such as the proximal face 3150 of marker band 3116. Each compression gap preferably has a width measured in a circumferential direction at the proximal end that exceeds the width near the distal end of the compression gap. The axial depth of the compression gaps may be approximately equal, so that the distal ends of the compression gaps all align in a transverse plane that is approximately parallel with the proximal face 3150. Alternatively, as illustrated in FIG. 4D, the distal ends of the compression gaps may be aligned progressively such that they lie on an inclined plane that may be approximately parallel to the inclined distal face 3136.

Alternatively, as shown in the marker bands of FIGS. 4F-4I, one or more compression gaps may extend between a proximal transverse face 3150 and a distal inclined face 3136, but not intersect the proximal transverse face 3150 (in contrast, for example, to the marker band of FIG. 4E). As described above, each compression gap 3176, 3178, 3180 may have a width measured in a circumferential direction at the proximal end that exceeds the width near the distal end of the compression gap, as shown in FIGS. 4H-4I. Alternatively, each compression gap 3170, 3172, 3174 may have a width measured in a circumferential direction at the proximal end that substantially equals or is substantially similar to the width near the distal end of the compression gap, as shown in FIGS. 4F-4G. An angle and/or shape of the distal end 3170a, 3174a, 3176a, 3180a of compression gaps 3170, 3174, 3176, 3180, respectively, may substantially equal or be similar to the angle of the distal face 3136, as described above and shown in FIGS. 4F-4I. The circumferentially continuous proximal transverse face 3150 of each of the embodiments in FIGS. 4F-4I is such that it enables securing to the coil, as described elsewhere herein and below.

In addition to or as an alternative to the tension element, any of the marker bands disclosed herein may be secured to the coil such as by adhesives, welding, or mechanical interference fit. In one mechanical interference fit implementation, a helical slot may be formed in the proximal sidewall of the marker band, extending circumferentially through at least about 45 degrees, and in some implementations at least about 180 degrees or about 360 degrees or more. This allows the distal end of the helical coil to be screwed into the helical slot in the marker band side wall while preserving the ID of the lumen and OD of the catheter across the joint.

As a further alternative, one or more tension elements maybe integrally formed with the marker band, such as by laser cutting the marker band and an elongate, proximally extending axial or helical strut tension element from a single tube stock.

The tension element may take the form of at least one and optionally at least two or four or 10 or more struts, which may extend proximally in a linear, spiral, or intersecting e.g., diamond pattern.

For example, the marker band in FIG. 4E (with optional compression gaps omitted for simplicity) includes a tension element in the form of a plurality of intersecting struts 3166 defining a tubular body having a plurality of sidewall openings 3168, which may progressively increase or decrease in compressibility in the proximal direction. The marker band and associated tension element struts 3166 may be slip fit over the tie layer with the coil wrapped around the outside of at least a portion of the length of the tension elements, with or without application of an adhesive prior to wrapping the coil. Alternatively, a plurality of proximal apexes may be formed in alignment on a transverse plane or other geometry that is complementary to the geometry of the distal end of the support structure (e.g., coil) in the catheter shaft, and be welded together end to end to provide a secure joint. Further, any of the embodiments of FIGS. 4A-4I may or may not include an axially extending slit, as described elsewhere herein to

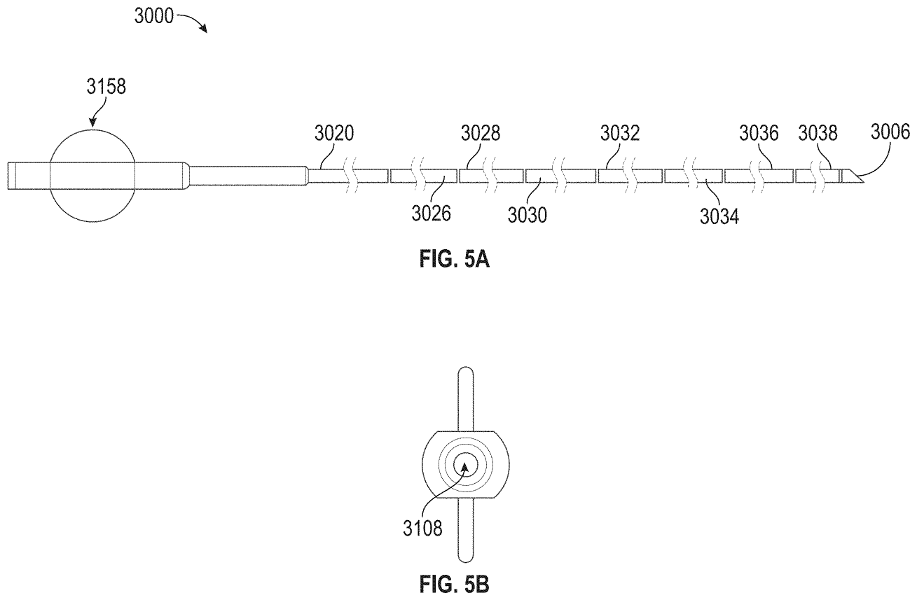

Referring to FIGS. 5A-5B, there is illustrated one example of an outer jacket segment stacking pattern for a progressive flexibility catheter of the type discussed in connection with FIG. 2. In some embodiments, a distal segment 3038 may have a length within the range of about 1 cm to about 3 cm , and a durometer of less than about 35 D or about 30 D. An adjacent proximal segment 3036 may have a length within the range of about 4 cm-6 cm, and a durometer of less than about 35 D or about 30 D. An adjacent proximal segment 3034 may have a length within the range of about 4 cm to about 6 cm, and a durometer of about 35 D or less. An adjacent proximal segment 3032 may have a length within the range of about 1 cm to about 3 cm, and a durometer within the range of from about 35 D to about 45 D (e.g., 40 D). An adjacent proximal segment 3030 may have a length within the range of about 1 cm to about 3 cm, and a durometer within the range of from about 50 D to about 60 D (e.g., about 55 D). An adjacent proximal segment 3028 may have a length within the range of about 1 cm to about 3 cm, and a durometer within the range of from about 35 D to about 50 D to about 60 D (e.g., about 55 D). An adjacent proximal segment 3026 may have a length within the range of about 1 cm to about 3 cm, and a durometer of at least about 60 D and typically less than about 75 D. More proximal segments may have a durometer of at least about 65 D or 70 D. The distal most two or three segments may comprise a material such as Tecothane™ or Neusoft™, and more proximal segments may comprise PEBAX™, Vestamid®, Grilamid® or other catheter jacket materials known in the art. At least three or five or seven or nine or more discrete segments may be utilized, having a change in durometer between highest and lowest along the length of the catheter shaft of at least about 10 D, preferably at least about 20 D and in some implementations at least about 30 D or about 40 D or more.

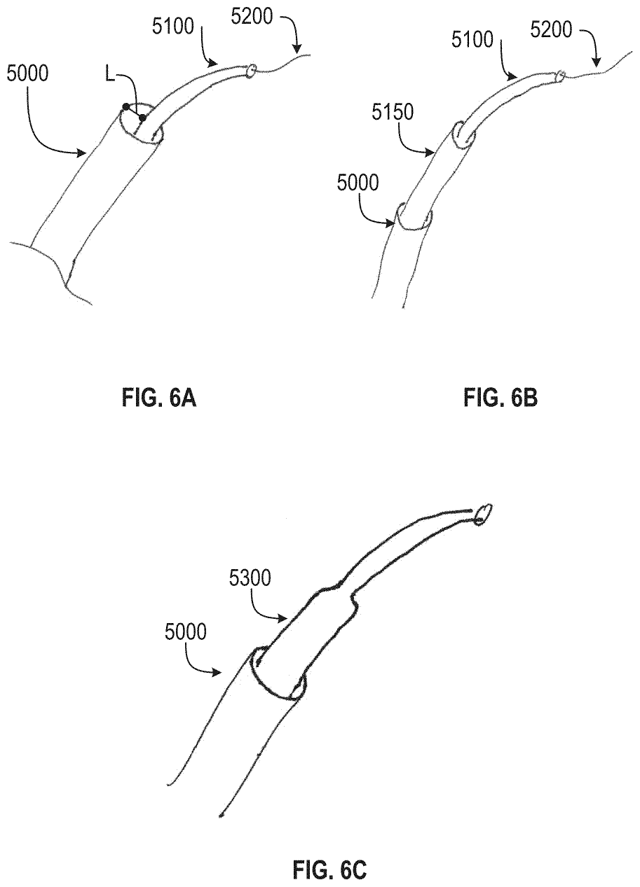

Referring to FIGS. 6A-17, various examples of an obturator are illustrated, according to various embodiments. Unless otherwise noted, each embodiment of an obturator as shown in FIGS. 6A-17 and/or as described herein may include components that are the same as or generally similar to the components in the figures illustrated and discussed herein. It will be understood that any of the obturators shown in FIGS. 6A-17 can be used with any of the devices and/or systems described and/or contemplated herein. It will also be understood that any of the devices and/or system described and/or contemplated herein can be modified to be used with any of the obturators shown in FIGS. 6A-17 or described herein. As with all embodiments in this specification, any feature, structure, material, method, or step that is described and/or illustrated in the various embodiments of the obturators of FIGS. 6A-17 and/or described herein can be used with or instead of any feature, structure, material, method, or step that is described and/or illustrated in any other device and/or system of this specification.

The obturator may be utilized to simplify and/or facilitate the approach for advancement of one or more aspiration catheter to the face of a clot. The obturator may replace the need for at least one of an additional microcatheter or an additional separate access catheter when accessing a clot with an aspiration catheter during a removal procedure. As explained in further detail herein, in some instances, the obturator may be configured to reduce a “ledge effect”—or reduce a rapid change in diameter between one or more catheters. FIGS. 6A-6C illustrate examples of this “ledge effect” and a benefit associated with use of an obturator. A reduction in the ledge effect may readily facilitate navigation of one or more catheters across a tortuous or particularly difficult area of the vasculature (e.g., the ophthalmic origin) and, thereby, facilitating advancement of an aspiration catheter to a clot. In some instances, the obturator may be configured to at least partially cross a clot and to deliver a clot retrieval mechanism (e.g., a stent retriever) distal to the clot.

By way of example, FIG. 6A illustrates a rapid change in diameter, or ledge L, which may typically occur between an aspiration catheter 5000 and an access catheter 5100 that is tracked over a guidewire 5200 to facilitate placement of the aspiration catheter 5000 adjacent to a clot. The ledge L occurs due to the discrepancy in diameter between the larger diameter aspiration catheter 5000—which relies on the larger cross-sectional area to provide a greater aspiration area—and the smaller diameter access catheter 5100—which relies on the smaller diameter to increase trackability over the guidewire and facilitate passage through the vasculature. As the access catheter 5100 and aspiration catheter 5000 are tracked through the vasculature, the ledge L may cause the aspiration catheter 5000 to get caught along a vessel wall (e.g., along a bend in the vasculature). FIG. 6B illustrates a current method of reducing the ledge L that relies upon an intermediate access catheter 5150 to provide a transition size between the diameters of the access catheter 5100 and the aspiration catheter 5000. However, this method increases the complexity of the procedure and increases time required to access the clot with the aspiration catheter 5000 since the separate catheter must be inserted into the vessel, tracked over the access catheter 5100, and removed from the vessel once the aspiration catheter 5000 reaches the clot prior to beginning aspiration. FIG. 6C illustrates an example of a method of utilizing an obturator 5300 as an access catheter, as described in further detail herein, that comprises a microcatheter segment and a dilator segment to facilitate tracking over a guidewire and passage through the vessel, while also reducing any ledge effect caused by a large discrepancy between the diameter of the aspiration catheter 5000 and a smaller access catheter. The obturator 5300, in some instances, can also reduce the complexity of the aspiration procedure and reduce the time associated with the procedure by avoiding necessity of utilizing a separate, intermediate access catheter.

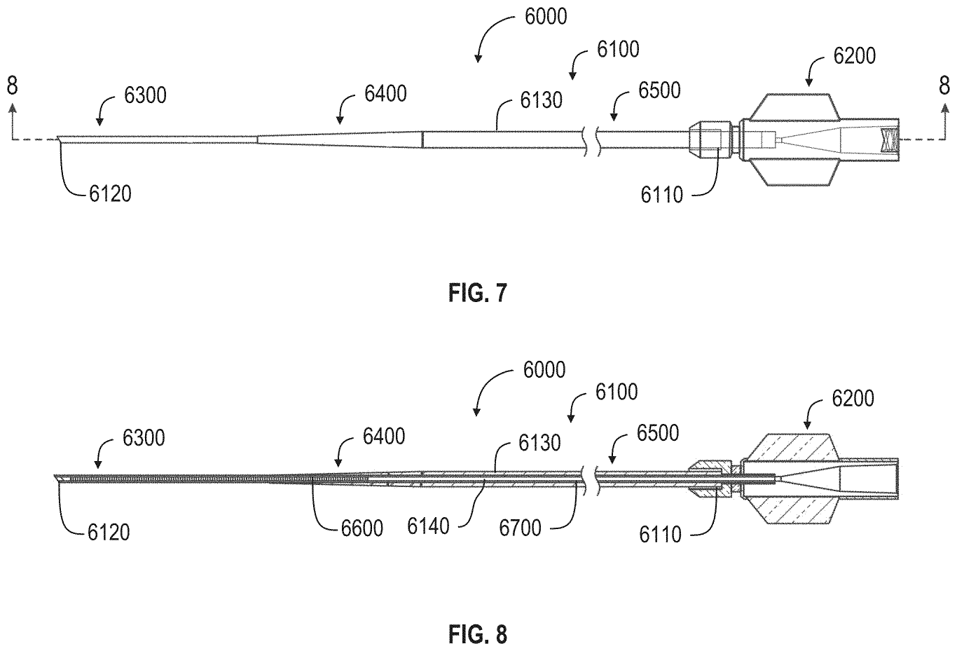

FIGS. 7 and 8 are various views of an obturator 6000, according to some embodiments. In particular, FIG. 7 is a side view of an obturator 6000 and FIG. 8 is a side cross-sectional view of the obturator 6000 of FIG. 7 taken along line 7 (line 7 shown in FIG. 8). Unless otherwise noted, the obturator 6000 as shown in FIGS. 7 and 8 may include components that are the same as or generally similar to the components in the figures illustrated and discussed herein. It will be understood that the obturator 6000 shown in FIGS. 7 and 8 can be used with any of the embodiments described and/or contemplated herein. It will also be understood that any of the embodiments described and/or contemplated herein can be modified to be used with the obturator 6000 shown in FIGS. 7 and 8. As with all embodiments in this specification, any feature, structure, material, method, or step that is described and/or illustrated in the embodiment of FIGS. 7 and 8 can be used with or instead of any feature, structure, material, method, or step that is described and/or illustrated in any other embodiment of this specification.

The obturator 6000 can include an elongate, flexible tubular body 6100 being attached to a hub 6200 at a proximal end 6110 of the tubular body 6100. The tubular body 6100 may further include a distal end 6120 and a sidewall 6130. The sidewall 6130 can at least partially defining a central lumen 6140 that extends axially through the tubular body 6100. It will be understood that the tubular body 6100 may include any feature, structure, or material that is described and/or illustrated in connection with the various embodiments of braids described herein (such as tubular body 16 or tubular body 3112).

The obturator 6000 can include one or more segments that each provide various functionalities to assist in the function of the obturator 6000. A distal end portion of the obturator 6000, in some instances, may comprise one or more of a microcatheter segment 6300, a dilator segment 6400, or a shaft segment 6500. The microcatheter segment 6300 can extend in a proximal direction from the distal end 6120 of the tubular body 6100. The shaft segment 6500 can extend in a distal direction from proximal end 6110 of the tubular body 6100. The dilator segment 6400 may be positioned on the tubular body 6100 at least partially in between the microcatheter segment 6300 and the shaft segment 6500. In some instances, one or more of the segments 6300, 6400, 6500 may overlap and/or otherwise structurally interact with any of the other segments 6300, 6400, 6500 throughout the length of the obturator 6000. In some embodiments, one or both of the microcatheter segment 6300 and the dilator segment 6400 may have kink resistance down to approximately an about 1 mm radius or larger without any lumen collapse. Further, the kink resistance minimum in the shaft segment 6500 may be increased to an approximately 20 mm radius or greater. In some embodiments, kink resistance at mid-shaft (approximately 20 cm from the distal end 6120) may be an approximately 2.5 mm radius or greater.

A length of the tubular body 6100 and/or of any one of the microcatheter segment 6300, the dilator segment 6400, and the shaft segment 6500 may be different depending upon the features desired in a particular product. The overall length of the tubular body may be within a range of about 135 cm to about 165 cm. For example, the length of the tubular body 6100 may be about 145 cm, about 150 cm, about 155 cm, about 160 cm, or about 165 cm, each +/− about 5 cm.

The length of the shaft segment 6500 can be significantly greater than the lengths of each of the microcatheter segment 6300 or the dilator segment 6400. The length of the shaft segment 6500, in some instances, may be about 70% to about 97% of the overall length of the tubular body 6100. For example, the shaft segment 6500 may be about 90% to about 97% of the length of the tubular body 6100. In some embodiments, the length of the shaft segment 6500 may be within a range of about 120 cm to about 160 cm. The length of the shaft segment 6500 can be at least about 140 cm, and in some embodiments, the length can be about 142 cm.

The length of the microcatheter segment 6300 can be any size configured to assist in a function of the microcatheter segment 6300. For example, in some instances described herein, the microcatheter segment 6300 may be configured to at least partially cross a clot in a vessel to assist in the removal of the clot. Therefore, the length of the microcatheter segment 6300 may be determined based on historical data of a clot length to permit the microcatheter segment 6300 to at least partially cross a clot such that distal end 6120 of the tubular body 6100 fully or partially crosses a clot in the vessel. In some instances, the length may be about 1% to about 5% or to about 7% of the overall length of the tubular body 6100. For example, the microcatheter segment 6300 may be about 2% to about 2.5% of the length of the tubular body 6100. In embodiments intending to fully cross the clot, a microcatheter segment 6300 may be sized to be only slightly longer than the length of the clot that it is disposed across. The microcatheter segment 6300 length may be based on historical data of clot length and in so doing have a length only slightly longer than the typical clot length. For example, if a typical clot length is found to be about 1.34 cm (0.53 inches), then a suitable microcatheter segment 6300 may be about 2.25 cm (0.89 inches). By closely matching the length of the clot, increased support across the clot is experienced due to the increased proximity of a dilator segment 6400 that, in some embodiments, may be stiffer than the microcatheter segment 6300. In further embodiments utilized for delivery, the increased support across the clot may facilitate delivery of devices to or beyond the clot. Further, in some embodiments, the length of the microcatheter segment 6300 may be within a range of about 2 cm to about 10 cm. For example, the length of the microcatheter segment 6300 may be about 4 cm to about 6 cm or about 3 cm to about 5 cm. The length of the microcatheter segment 6300, in some instances, may be about 2 cm, about 3.5 cm, about 4 cm, or about 6 cm. The length of the microcatheter segment 6300 can be at least about 1 cm or at least about 2 cm. In some embodiments, the microcatheter segment 6300 may comprise a first proximal section of about 1 cm to about 5 cm and may comprise a second distal section of about 0.5 cm to about 2.5 cm. For example, the first proximal section may be of about 1 cm to about 3 cm and the second distal section may be about 1.75 cm to about 2.25 cm.

The length of the dilator segment 6400 can be any size configured to assist in a function of the dilator segment 6400. For example, in some instances described herein, the dilator segment 6400 may be provide a transition zone between a smaller diameter of the microcatheter segment 6300 and a larger diameter of the shaft segment 6500. Therefore, the length of the dilator segment 6400 may be determined based on a desired angle of taper relative to the diameters of each of the microcatheter segment 6300 and the shaft segment 6500. In some instances, the dilator segment 6400 may be about 0.5% to about 10% of the overall length of the tubular body 6100. For example, the dilator segment 6400 may be about 0.5%, about 2% or about 2.3% of the length of the tubular body 6100. In some embodiments, the length of the dilator segment 6400 may be within a range of about 0.5 cm to about 10 cm. For example, the length of the dilator segment 6400 may be 0.5 cm to about 1.5 cm, about 0.5 cm to about 3 cm, about 4 cm to about 6 cm or about 3 cm to about 5 cm. By way of another example, the length of the dilator segment 6400 may be about 3 cm to about 6 cm. The length of the dilator segment 6400, in some instances, may be about 3.5 cm. The length of the dilator segment 6400 can be at least about 0.5 cm, at least about 1 cm, at least about 2 cm, or at least about 3 cm. The taper angle along the length of the dilator segment 6400 may be at about 1% to about 5%, about 2% to about 4%, or about 2% to about 3%.

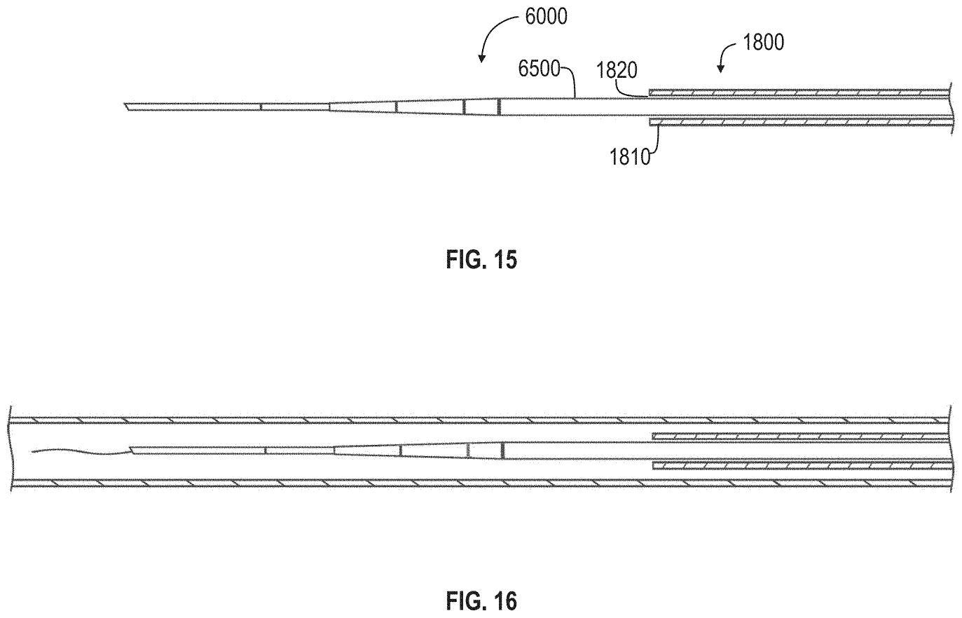

An outer diameter of the shaft segment 6500 can be any size configured to assist in a function of the shaft segment 6500. For example, in some instances described herein, the shaft segment 6500 may be configured to at least partially reduce a “ledge effect”—or a rapid change in diameter—between the obturator 6000 and an aspiration catheter that is tracked over the obturator. A reduction in the ledge effect may readily facilitate navigation of the aspiration catheter across a tortuous or particularly difficult area of the vasculature (e.g., the ophthalmic origin) and, thereby, facilitating advancement of an aspiration catheter to a clot. For example, as illustrated in FIG. 15, an outer diameter of the shaft segment 6500 may be sized to reduce a ledge or gap 1820 between the outer diameter of the shaft segment 6500 and an inner diameter of a distal end 1810 of a separate catheter 1800 (such as an aspiration catheter). The reduction in a size of the gap 1820 can advantageously assist in preventing the catheter 1810 from becoming lodged against a vessel wall, particularly along a curvature of the vessel, as the catheter 1800 is tracked over the obturator 6000.

The outer diameter of the shaft segment 6500 may be determined based on general data of an inner diameter of a separate outer catheter to permit a reduction in the ledge or gap. In some instances, an outer diameter of the shaft segment 6500 may be at least about 90% of an inner diameter of the separate catheter to be tracked over the obturator 6000 (e.g., aspiration catheter 1800 as shown in FIG. 15). For example, the outer diameter of the shaft segment 6500 may be about 95% to about 97% (e.g., about 96%) of the inner diameter of the separate catheter. In some embodiments, an inner diameter of a separate outer catheter may be no more than about 0.00762 cm (0.003 inches) larger than the outer diameter of the shaft segment 6500. In some embodiments, the inner diameter of the separate outer catheter may be no more than about 0.0508 cm (0.02 inches) or, more specifically, no more than about 0.00254 cm (0.01 inches) larger than the outer diameter of the shaft segment 6500. In some embodiments, the outer diameter of the shaft segment 6500 may be within a range of about 0.127 cm (0.05 inches) to about 0.23 cm (0.09 inches). In some embodiments, the outer diameter of the shaft segment 6500 may be about 0.127 cm (0.05 inches) to about 0.178 cm (0.07 inches) or, more specifically, may be about 0.1397 cm (0.055 inches). In some embodiments, the outer diameter of the shaft segment 6500 may be about at least about 0.127 cm (0.05 inches), at least about 0.1397 cm (0.055 inches), and/or to about 0.160 cm (0.063 inches). In a further example, the outer diameter of the shaft segment 6500 may be about 0.170 cm (0.067 inches) to about 0.221 cm (0.087 inches) depending on the size of the intended separate outer aspiration catheter. The outer diameter of the shaft segment 6500, in some instances, may be about 0.216 cm (0.085 inches) if a separate catheter with an inner diameter of about 0.224 cm (0.088 inches) is used with the obturator 6000. In some embodiments, the outer diameter of the shaft segment 6500 can be at least about 0.20 cm (0.08 in).

In some instances, an outer diameter of the shaft segment 6500 may be at least about two times as large—or at least about three or four or five times as large as an outer diameter of the microcatheter segment 6300. For example, the outer diameter of the shaft segment 6500 may be about 150% to about 500% (e.g., at least about 170%, at least about 240%, or at least about 325%) as large as the outer diameter of the microcatheter segment 6300. The shaft segment 6500 can comprise the largest diameter of the tubular body 6100.

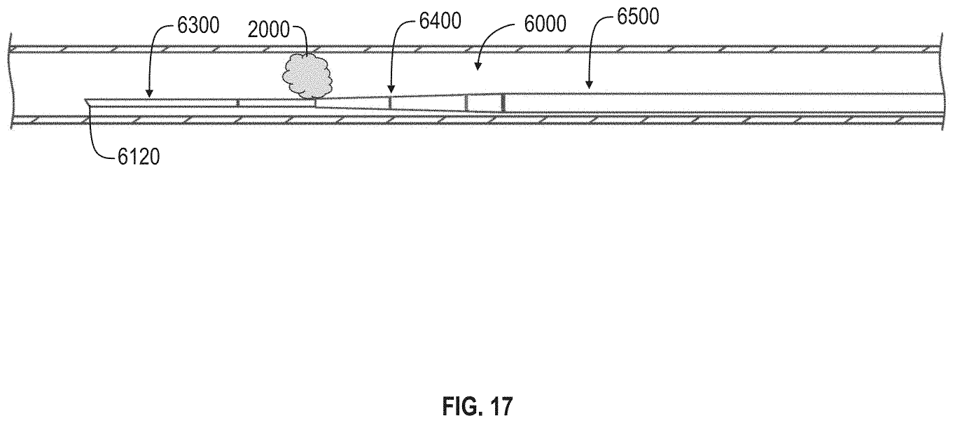

An outer diameter of the microcatheter segment 6300 can be any size configured to assist in a function of the microcatheter segment 6300. In some embodiments, outer diameter of the microcatheter segment 6300 may range from about 0.064 cm (0.025 inches) to about 0.178 cm (0.07 inches). For example, the outer diameter may be within a range of about 0.076 cm (0.03 inches) to about 0.152 cm (0.06 inches) or about 0.089 cm (0.035 inches) to about 0.14 (0.055 inches), with the distal most end being the narrowest and the proximal most end being the widest. For example, in some instances described herein, the microcatheter segment 6300 may be configured to at least partially cross a clot in a vessel to assist in the removal of the clot. For example, as illustrated in FIG. 17, an outer diameter of the microcatheter segment 6500 may be sized to cross through a clot 2000 and/or between a clot 2000 and a vessel wall 2100.

In some embodiments, the outer diameter of the microcatheter segment 6300 may be at least about 0.05 cm (0.020 inches) and/or no greater than about 0.1016 cm (0.040 inches). For example, the outer diameter of the microcatheter segment 6300 may be within the range of from about 0.066 cm (0.026 inches) to about 0.1016 cm (0.040 inches).

The outer diameter of the microcatheter segment 6300, in some instances, may generally increase from a distal section of the microcatheter segment 6300 towards a proximal section of the microcatheter segment 6300. A distal section of the microcatheter segment 6300 may comprise a smaller outer diameter (e.g., about 0.035 inches) relative to a proximal section of the microcatheter segment 6300 with a larger outer diameter (e.g., about 0.049 inches).

In some embodiments, the outer diameter of the dilator segment 6400 may generally increase from a distal section of the dilator segment 6400 towards a proximal section of the dilator segment 6400. For example, the distal section of the dilator segment 6400 may include an outer diameter of no more than about 0.102 cm (0.040 inches) and the proximal section of the dilator segment 6400 may include an outer diameter of no more than about 0.178 cm (0.070 inches). The distal section and/or the proximal section may comprise any diameter or range of diameters as described herein in connection with the outer diameter of the dilator segment 6400. For example, the outer diameter of the distal section of the dilator segment 6400 may be at least about 0.03 cm (0.076 inches), increasing in outer diameter to at least about 0.05 cm (0.127 inches) over the distalmost about 2 cm to about 6 cm of the dilator segment 6400 and tapering up to at least about 0.06 cm (0.152 inches) at the proximal section of the dilator segment 6400.

The outer diameter of the dilator segment 6400 can be any size configured to assist in functioning as a transition zone between a smaller diameter of the microcatheter segment 6300 and a larger diameter of the shaft segment 6500. Therefore, the outer diameter of the dilator segment 6400 may vary along the length of the dilator segment 6400 based on a desired angle of taper relative to the diameters of each of the microcatheter segment 6300 and the shaft segment 6500. In some instances, the dilator segment 6500 may comprise multiple distinct tapered sections such that each section comprises various angles of taper relative to a longitudinal axis of the obturator 6000. For example, the dilator segment 6400 may comprise a first section with a first taper angle and a second section with a second taper angle such that the first taper angle is different than a second taper angle. The dilator segment 6400, in some instances, comprises a plurality of tapering sections and a plurality of tapering angles along the length of the dilator segment 6400.

An inner diameter of the tubular body 6100 (inside diameter of the central lumen 6140) can be any size configured to assist in a function of the central lumen 6140. In some instances, described herein, the central lumen 6140 may be configured to permit one or more additional devices and/or instruments through the central lumen 6140. For example, the central lumen 6140 may be configured to receive at least one of a guidewire and/or a stent retriever therethrough to permit passage of at least one of the guidewire and/or the stent retriever through an interion of the obturator 6000.

The diameter of the central lumen 6140 may be determined based on general data of a diameter or width of the separate device and/or instrument to permit passage therethrough. In some instances, the diameter of the central lumen 6140 may be at least about 0.051 cm (0.020 inches) to about 0.076 cm (0.030 inches). For example, the diameter of the central lumen 6140 may be about 0.066 cm (0.026 inches) (e.g., 0.067 cm (0.026 inches)) depending on the size of the intended separate device and/or instrument.

A wall thickness of the sidewall 6130 can be any size configured to assist in a function of the obturator 6000. For example, the wall thickness of the sidewall 6130 can be a function of the diameter of the central lumen 6140 relative to the outer diameter of any one of the microcatheter segment 6300, the dilator segment 6400, or the shaft segment 6500. In some instances, a wall thickness of the sidewall 6130 along the shaft segment 6500 can be significantly greater than the wall thicknesses of the sidewall 6130 along the microcatheter segment 6300 and/or the dilator segment 6400. The wall thickness of the sidewall 6130 along the shaft segment 6500 may be at least about two times as large—or at least about three times as large—as the wall thickness of the sidewall 6130 along the microcatheter segment 6300.

For example, the wall thickness of the sidewall 6130 along the shaft segment 6500 may be at least about 150% to about 1,200% (e.g., at least about 170%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%) as large as the wall thickness of the sidewall 6130 along the microcatheter segment 6300. The wall thickness of the sidewall 6130 along the shaft segment 6500 can comprise the largest wall thickness along the tubular body 6100. In some instances, the wall thickness of the sidewall 6130 along the shaft segment 6500 may be at least about 0.025 cm (0.010 inches) to about 0.051 cm (0.020 inches) or about 0.036 cm (0.014 inches) to about 0.15 cm (0.016 inches). For example, the wall thickness of the sidewall 6130 along the shaft segment 6500 may be about 0.06 inches. The wall thickness of the sidewall 6130 along the shaft segment 6500, in some embodiments, may be at least about 0.028 inches.

The wall thickness of the sidewall 6130 along the microcatheter segment 6300 can comprise the smallest wall thickness along the tubular body 6100. In some instances, the wall thickness of the sidewall 6130 along the microcatheter segment 6300 may be at least about 0.003 cm (0.001 inches) and/or no more than about 0.03 cm (0.01 inches). For example, the wall thickness of the sidewall 6130 along the microcatheter segment 6300 may be no more than about 0.005 cm (0.002 inches), no more than about 0.013 cm (0.005 inches), no more than about 0.01778 cm (0.007 inches), or no more than about 0.02 cm (0.008 inches).

The wall thickness of the sidewall 6130 along the dilator segment 6400 may generally increase as the outer diameter of the dilator segment 6400 generally increases from a distal end of the dilator segment 6400 towards a proximal end of the dilator segment 6400. The inside diameter of the central lumen may be substantially constant throughout each of the shaft segment 6500, dilator segment 6400 and microcatheter segment 6300. For example, a wall thickness of the sidewall 6130 along the dilator segment 6400 may generally increase from or vary between about 0.025 cm (0.01 inches) to about 0.051 cm (0.02 inches) or about 0.033 cm (0.013 inches) to about 0.041 cm (0.016 inches).

The obturator 6000 may comprise a coil 6600 (as shown in FIG. 8) embedded in the sidewall 6130 of the obturator 6000. It will be understood that the coil 6600 may include any feature, structure, or material that is described and/or illustrated in connection with the various embodiments of coils described herein (such as coil 3024 or coil 3122). The coil 6600 can be within the microcatheter segment 6300. In some instances, a distal end of the coil 6600 can be positioned proximate to the distal end 6120 of the obturator 6000 and can extend proximally towards the dilator segment 6400. The coil 6600, in some embodiments, can partially extend through at least a portion of and optionally entirely through the dilator segment 6400. The coil 6600, in some embodiments, can extend proximally into or at least partially through the shaft segment 6500 by between about 25 cm to about 60 cm or about 30 cm to about 40 cm or about 45 cm to about 60 or at least about 30 cm or at least about 40 cm or at least about 45 cm or at least about 50 cm.

The pitch of the coil 6600 may remain constant throughout the length of the coil 6600 or the pitch may transition as the coil 6600 extends in a distal direction. In some instances, the coil 6600 may have a smaller pitch along a distal portion of the coil 6600 relative to a larger pitch along a proximal portion of the coil 6600. The distal portion of the coil 6600 may comprise a pitch of between about 0.00762 cm (0.003 inches) to about 0.0127 cm (0.05 inches). For example, the outer diameter of the radiopaque marker may be about 0.01016 cm (0.04 inches). The proximal portion of the coil 6600 may comprise a pitch of between about 0.01016 cm (0.04 inches) to about 0.01524 cm (0.06 inches). For example, the outer diameter of the radiopaque marker may be about 0.0127 cm (0.05 inches). The proximal portion of the coil 6600 may be longer than the distal portion of the coil 6600.

The obturator 6000 may comprise a braid 6700 (as shown in FIG. 8) embedded in the sidewall 6130 of the obturator 6000. It will be understood that the braid 6700 may include any feature, structure, or material that is described and/or illustrated in connection with the various embodiments of braids described herein (such as braid 3010 or braid 3122).

The braid 6700 can be within the shaft segment 6500. In some instances, a proximal end of the braid 6700 can be positioned proximate to the proximal end 6110 of the obturator 6000 and can extend distally towards the dilator segment 6400. The braid 6700, in some embodiments, can partially extend through at least portion of and optionally entirely through the dilator segment 6400. In some embodiments, a distal portion of the braid 6700 can overlap with or abut against a proximal portion of the coil 6600. The distal portion of the braid 6700 can extend over the proximal portion of the coil 6600 for at least about 2 cm, about 2 cm to about 10 cm, about 2 cm to about 8 cm, or about 4 cm to about 6 cm or more. Alternatively, the distal portion of the braid 6700 can extend under the proximal portion of the coil 6600.

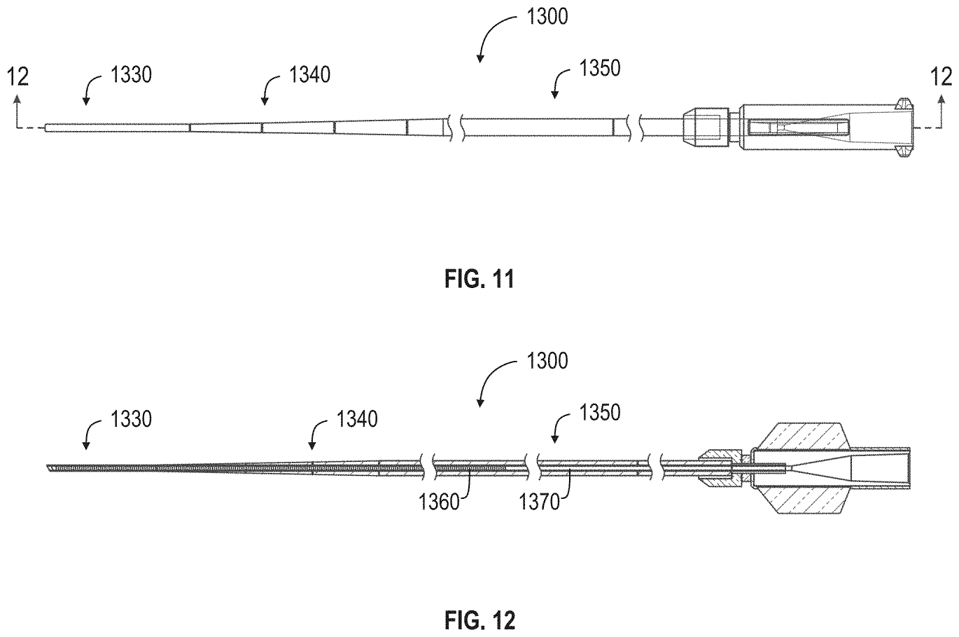

Referring to FIGS. 11 and 12, another example of an obturator 1300 is illustrated, according to another embodiment. Unless otherwise noted, the obturator 1300 as shown in FIGS. 11 and 12 may include components that are the same as or generally similar to the components in connection with any other embodiment of an obturator as illustrated and/or discussed herein. In some instances, an obturator 1300 may comprise a braid 1370 that resides entirely within the shaft segment 1350. A coil 1360 may extend from the microcatheter segment 1330 to the shaft segment 1350 such that the braid 1370 overlaps with and/or abuts against the coil 1360 within the shaft segment 1350.

One or more portions of the obturator 6000 may comprise a radiopaque structure and/or radiopaque properties configured to facilitate visualization of the one or more portions of the obturator 6000 with an imaging device. In some instances, the obturator 6000 may comprise a radiopaque marker at or proximate to the distal end 6120. It will be understood that the radiopaque marker of the obturator may include any feature, structure, or material that is described and/or illustrated in connection with the various embodiments of radiopaque markers described herein (such as marker 3040).

In some instances, the radiopaque marker may comprise a tubular shape such that at least one of a proximal face of the radiopaque marker or a distal face of the radiopaque marker extends along a plane that is generally perpendicular to a longitudinal axis of the obturator. The radiopaque marker may comprise an outer diameter of between about 0.0635 cm (0.025 inches) to about 0.09779 cm (0.0385 inches). For example, the outer diameter of the radiopaque marker may be about 0.07874 cm (0.031 inches) to about 0.08128 cm (0.032 inches) or, more specifically, about 0.08001 cm (0.0315 inches). The radiopaque marker may comprise an inner diameter of between about 0.0508 cm (0.020 inches) to about 0.0889 cm (0.035 inches). For example, the inner diameter of the radiopaque marker may be about 0.07366 cm (0.029 inches) to about 0.0762 cm (0.03 inches) or, more specifically, about 0.07493 cm (0.0295 inches). The radiopaque marker may comprise a wall thickness of between about 0.002032 cm (0.0008 inches) to about 0.003048 cm (0.0012 inches). For example, the wall thickness of the radiopaque marker may be about 0.002413 cm (0.00095 inches) to about 0.00381 cm (0.0015 inches) or, more specifically, about 0.00254 cm (0.001 inches). The radiopaque marker may comprise a length of between about 0.0381 cm (0.015 inches) to about 0.0889 cm (0.035 inches). For example, the length of the radiopaque marker may be about 0.0508 cm (0.02 inches) to about 0.0762 cm (0.03 inches) or, more specifically, about 0.0635 cm (0.025 inches).

At least a portion of dilator segment 6400, in some embodiments, may comprise one or more radiopaque markers or dopants. It will be understood that the radiopaque marker or dopant may include any feature, structure, or material that is described and/or illustrated in connection with the various embodiments of radiopaque markers described herein (such as marker 3040). A radiopaque dopant and/or marker may be positioned at least at a distal end of the dilator segment 6400. The dopant and/or marker can, in some instances, facilitate visualization of a transition between the microcatheter segment 6300 and the dilator segment 6400. In some embodiments, the dilator segment 6400 comprises a radiopaque dopant along an entire length of the dilator segment 6400.

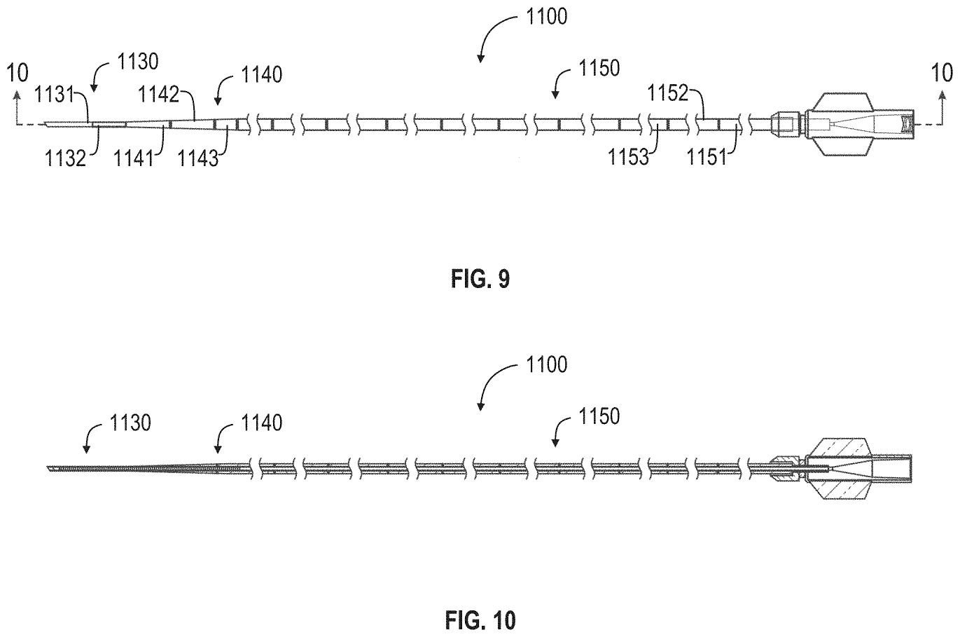

Referring to FIGS. 9 and 10, another example of an obturator 1100 is illustrated, according to another embodiment. Unless otherwise noted, the obturator 1100 as shown in FIGS. 9 and 10 may include components that are the same as or generally similar to the components in connection with any other embodiment of an obturator as illustrated and/or discussed herein. An obturator 1100 may comprise a microcatheter segment 1130, a dilator segment 1140, and a shaft segment 1150, which may be the same or generally similar to any corresponding segment described in connection with any other embodiment of an obturator described herein. In some instances, one or more of each of the segments 1130, 1140, 1150 can comprise a plurality of sections that are each part of one of the segments 1130, 1140, 1150. For example, the microcatheter segment 1130 may comprise a first section 1131, which may have a length within the range of about 1 cm to about 3 cm, and a durometer of less than about 20 D, and a second section 1132, which may have a length within the range of about 0.5 cm to about 2.5 cm, and a durometer of less than about 20 D. By way of another example, the dilator segment 1140 may comprise a first section 1141, which may have a length within the range of about 0.25 cm to about 10.75 cm, and a durometer of about 15 D to about 25 D, a second section 1142, which may have a length within the range of about 2 cm to about 4 cm, and a durometer less than about 20 D, and a third section 1143, which may have a length within the range of about 4 cm to about 6 cm, and a durometer within the range of from about 20 D to about 30 D. By way of another example, the shaft segment 1150 may comprise a plurality of sections (e.g., a first section 1151, a second section 1152, a third section 1153, etc.). It will be understood that the number of sections within each segment 1130, 1140, 1150 may be altered based on the particular configuration of the obturator 1100 and are not limited to any particular number of sections that are illustrated in FIGS. 9 and 10. Each of the sections within the respective segments 1130, 1140, 1150 may comprise varying characteristics relative to one or more of the other sections within the respective segment. For example, a first section may comprise one or more characteristics that differ from a second section. The characteristics that may be altered include, but are not limited to, at least one or outer diameter, inner diameter, length, wall thickness, durometer, durability, flexibility, material, or radiopacity.

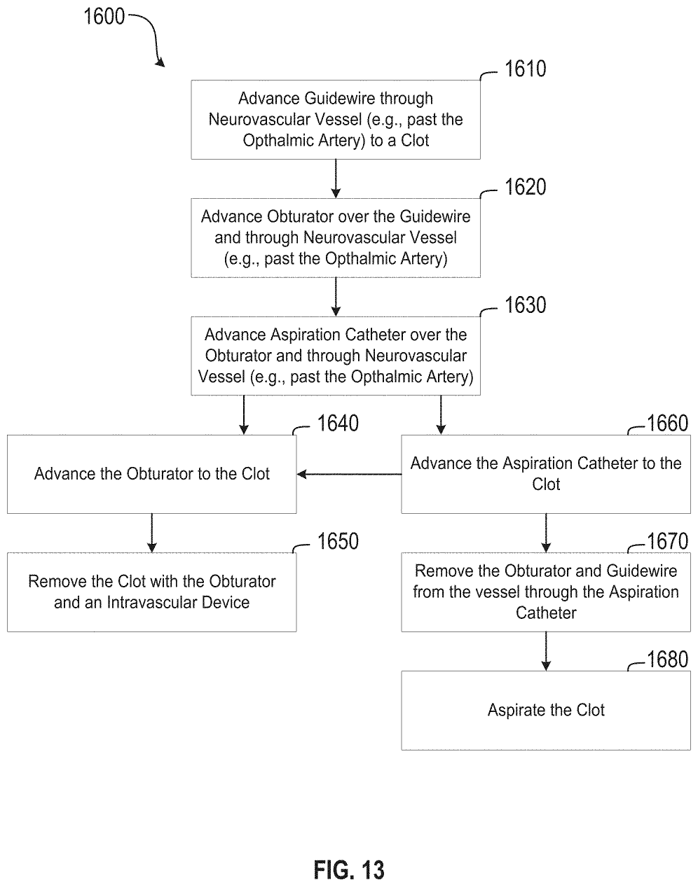

Referring to FIG. 13, an example method 1600 of treating a patient using an obturator is illustrated. It will be understood that any of the obturators shown in FIGS. 6A-17 and any of the devices and system described herein can be used with the method and steps described and/or contemplated herein. Additionally, any methods disclosed herein need not be performed in the order recited and need not perform every step disclosed in the method. The methods disclosed herein include certain actions taken by a practitioner; however, they can also include any third-party instruction of those actions, either expressly or by implication. Alternatively, the methods disclosed herein may, at least in part, be performed by an automated or robotic system.

At step 1610, the method includes advancing a guidewire through one or more vessels to a clot. The guidewire may be advanced past one or more or across a tortuous or particularly difficult area of the vasculature (e.g., the ophthalmic artery).

At step 1620, the method includes advancing an obturator over the guidewire and through the one or more vessels. The obturator may be advanced past one or more or across a tortuous or particularly difficult area of the vasculature (e.g., the ophthalmic artery). The obturator may be advantageously configured to facilitate passage through the difficult vasculature. In one implementation, the microcatheter extends beyond the clot, and the obturator is positioned with the clot at the transition between the microcatheter and the dilator.

At step 1630, the method includes advancing an aspiration catheter over the obturator and through the one or more vessels. The obturator may facilitate advancement of the aspiration catheter past one or more or across a tortuous or particularly difficult area of the vasculature by reducing a “ledge” effect or gap that would otherwise be present during use of a different access catheter. Minimizing the gap present between the obturator and the aspiration catheter reduces the effect that the gap snags or gets caught on a vessel wall while navigating the vasculature.

As the aspiration catheter is advanced past any particularly difficult areas in the vasculature, it may be determined whether the clot will be removed through use of an intravascular device (e.g., a stent retriever).

If the clot location or composition is such that it needs to be removed using an intravascular device, then the obturator may be advanced over the guidewire to the clot at step 1640.

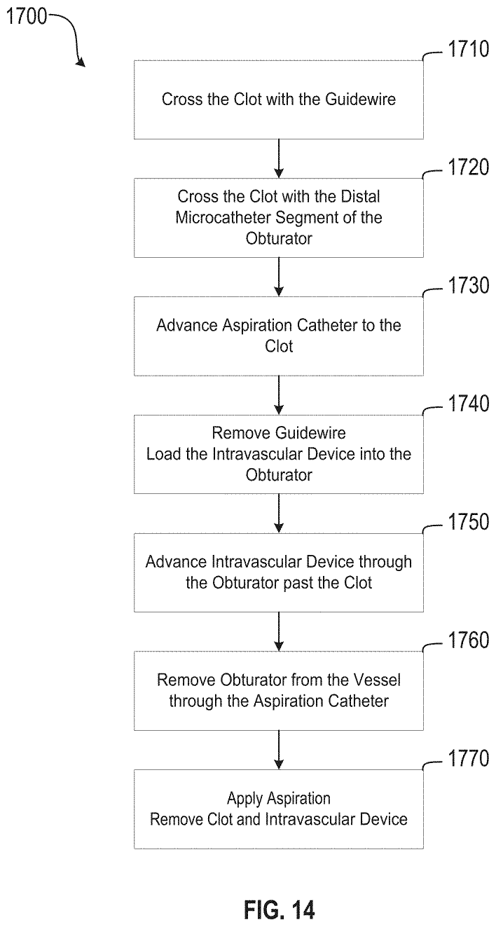

At step 1650, the method includes removing the clot with the obturator and the intravascular device. This step is further described in connection with method 1700 illustrated in FIG. 14 and described in further detail herein.

If the clot location or composition is such that an intravascular device is not needed, shown at step 1630, then the aspiration catheter may be advanced over the obturator to the clot at step 1660.

Once the aspiration catheter is advanced the clot, the method includes removing the obturator and the guidewire from the vessel through an interior of the aspiration catheter at 1670.

At step 1680, the method includes aspirating and/or removing the clot from the vessel using the aspiration catheter.

In some instances, shown at step 1660, aspiration alone may not be sufficient to remove the clot once the aspiration catheter is advanced to the clot. In this case, the method may include advancing the obturator to the clot at step 1640.

Referring to FIG. 14, an example method 1700 of treating a patient using an obturator is illustrated. It will be understood that any of the obturators shown in FIGS. 6A-17 and any of the devices and system described herein can be used with the method and steps described and/or contemplated herein. Additionally, any methods disclosed herein need not be performed in the order recited and need not perform every step disclosed in the method. The methods disclosed herein include certain actions taken by a practitioner; however, they can also include any third-party instruction of those actions, either expressly or by implication. Alternatively, the methods disclosed herein may, at least in part, be performed by an automated or robotic system.

Method 1700 provides further detail in connection with the removal of the clot utilizing the use of the obturator and the intravascular device. At step 1710, the method includes advancing the guidewire such that the guidewire at least reaches and optionally crosses the clot. It is noted that step 1710 is optional and method 1700 may begin at step 1720.

At step 1720, the method includes advancing the obturator such that a microcatheter segment of the obturator at least partially crosses through the clot or between the clot and a vessel wall. FIG. 17 illustrates an example positioning of the microcatheter segment 6300 relative to the clot 2000. In some instances, one or more radiopaque markers and/or dopants located along the obturator 6000 can facilitate precise placement of the microcatheter segment 6300 along the clot 2000. For example, it may be advantageous to position the clot 2000 at a transition location between the microcatheter segment 6300 and the dilator segment 6400.

At step 1730, the method includes advancing the aspiration catheter along the obturator to the clot.

At step 1740, the method includes removing the guidewire from the vessel through the obturator and loading an intravascular device (e.g., a stent retriever) into the obturator.

At step 1750, the method includes advancing the intravascular device through the obturator and past the clot.

At step 1760, the method includes removing the obturator form the vessel through the aspiration catheter.

At step 1770, the method includes applying aspiration and removing the clot and the intravascular device from the vessel and into the aspiration catheter.

### Example Embodiments

An access catheter comprising one or more of the following:

an elongate, flexible tubular body comprising one or more of the following:
a proximal end,
a distal end, and
a side wall at least partially defining a central lumen, the central lumen extending axially therethrough;

a distal microcatheter segment extending proximally from the distal end, the distal microcatheter segment comprising one or more of the following:
a length of about 2 cm to about 10 cm, and
an outer diameter of no more than about 0.04 inches;

a proximal shaft segment extending distally from the proximal end, the proximal shaft segment comprising one or more of the following:
a length of at least about 140 cm; and
an outer diameter of at least about 0.055 inches; and

a tapered dilator segment being positioned in between the distal microcatheter segment and the proximal shaft segment.

An access catheter of any embodiment described herein, further comprising a coil being in the side wall, the coil extending proximally through the distal microcatheter segment and at least partially through the tapered dilator segment.

An access catheter of any embodiment described herein, further comprising a tubular braid in the side wall, the tubular braid extending distally from the proximal end.

An access catheter of any embodiment described herein, wherein at least a portion of the tubular braid extends over a proximal end of the coil.

An access catheter of any embodiment described herein, further comprising a radiopaque marker at the distal end.

An access catheter of any embodiment described herein, wherein at least a distal end of the tapered dilator segment is visible under fluoroscopy.

An access catheter of any embodiment described herein, wherein the tapered dilator segment comprises a radiopaque dopant.

An access catheter of any embodiment described herein, further comprising a radiopaque marker being positioned adjacent a transition between the tapered dilator segment and the distal microcatheter segment.

An access catheter of any embodiment described herein, wherein a wall thickness of the distal microcatheter segment is no more than about 0.007 inches.

An access catheter of any embodiment described herein, wherein a wall thickness of the proximal shaft segment is at least about 0.01 inches.

An access catheter of any embodiment described herein, wherein a length of the tubular body between the distal microcatheter segment and the proximal shaft segment is between about 0.5 cm and about 2.5 cm.

An access catheter of any embodiment described herein, wherein the distal microcatheter segment comprises one or more of the following:

a distal section comprising an outer diameter of no more than about 0.040 inches; and

a proximal section comprising an outer diameter of no more than about 0.070 inches.

A method of treating a patient, the method comprising one or more of the following steps:

advancing a microcatheter segment of an access catheter across a clot; and

advancing an aspiration catheter over the access catheter;

wherein an inner diameter of the aspiration catheter is no more than about 0.01 inches larger than an outer diameter of the access catheter.

A method of any embodiment described herein, further comprising advancing an intravascular device through the access catheter.

A method of any embodiment described herein, wherein the intravascular device comprises a stent retriever.

An access system for crossing a clot and navigating an aspiration catheter beyond an ophthalmic artery, the access system comprising one or more of the following:

an access catheter comprising a microcatheter distal section and an obturator proximal section; and

an aspiration catheter advanceable over the access catheter;

wherein a sliding fit between a distal end of the aspiration catheter and the access catheter minimizes a ledge at the distal end of the aspiration catheter.

An access system of any embodiment described herein, wherein the microcatheter distal section comprises an outer diameter of no more than about 0.040 inches.

An access system of any embodiment described herein, wherein the aspiration catheter comprises an outer diameter of at least about 0.070 inches.

A method of treating a patient, the method comprising one or more of the following steps:

providing an access catheter, the access catheter comprising:

a distal microcatheter section comprising an outer diameter of no more than about 0.040 inches and a length of about 2 cm to about 10 cm, and

a proximal shaft comprising an outer diameter of at least about 0.055 inches; and

advancing the distal microcatheter section across a clot.

A method of any embodiment described herein, further comprising advancing an aspiration catheter over the access catheter.

A method of any embodiment described herein, further comprising advancing an intravascular device through the access catheter.

A method of any embodiment described herein, wherein the intravascular device is a stent retriever.

What is claimed is:

1. An access catheter comprising:
an elongate, flexible tubular body comprising:
a proximal end,
a distal end, and
a side wall at least partially defining a central lumen, the central lumen extending axially therethrough;
a distal microcatheter segment extending proximally from the distal end, the distal microcatheter segment comprising:
a length of about 2 cm to about 10 cm, and
an outer diameter of no more than about 0.04 inches;
a proximal shaft segment extending distally from the proximal end, the proximal shaft segment comprising:
a length of at least about 140 cm; and
an outer diameter of at least about 0.055 inches; and
a tapered dilator segment being positioned in between the distal microcatheter segment and the proximal shaft segment.

2. An access catheter as in claim 1, further comprising a coil being in the side wall, the coil extending proximally through the distal microcatheter segment and at least partially through the tapered dilator segment.

3. An access catheter as in claim 2, further comprising a tubular braid in the side wall, the tubular braid extending distally from the proximal end.

4. An access catheter as in claim 3, wherein at least a portion of the tubular braid extends over a proximal end of the coil.

5. An access catheter as in claim 1, further comprising a radiopaque marker at the distal end.

6. An access catheter as in claim 1, wherein at least a distal end of the tapered dilator segment is visible under fluoroscopy.

7. An access catheter as in claim 6, wherein the tapered dilator segment comprises a radiopaque dopant.

8. An access catheter as in claim 6, further comprising a radiopaque marker being positioned adjacent a transition between the tapered dilator segment and the distal microcatheter segment.

9. An access catheter as in claim 1, wherein a wall thickness of the distal microcatheter segment is no more than about 0.007 inches.

10. An access catheter as in claim 9, wherein a wall thickness of the proximal shaft segment is at least about 0.01 inches.

11. An access catheter as in claim 1, wherein a length of the tubular body between the distal microcatheter segment and the proximal shaft segment is between about 0.5 cm and about 2.5 cm.

12. An access catheter as in claim 1 wherein the distal microcatheter segment comprises:
a distal section comprising an outer diameter of no more than about 0.040 inches; and
a proximal section comprising an outer diameter of no more than about 0.070 inches.

13. A method of treating a patient, the method comprising:
advancing a microcatheter segment of an access catheter across a clot; and
advancing an aspiration catheter over the access catheter;
wherein an inner diameter of the aspiration catheter is no more than about 0.01 inches larger than an outer diameter of the access catheter.

14. A method as in claim 13, further comprising advancing an intravascular device through the access catheter.

15. A method as in claim 14, wherein the intravascular device comprises a stent retriever.

16. A method of treating a patient, the method comprising:
providing an access catheter, the access catheter comprising:
a distal microcatheter section comprising an outer diameter of no more than about 0.040 inches and a length of about 2 cm to about 10 cm, and
a proximal shaft comprising an outer diameter of at least about 0.055 inches; and
advancing the distal microcatheter section across a clot.

17. A method as in claim 16, further comprising advancing an aspiration catheter over the access catheter.

18. A method as in claim 16, further comprising advancing an intravascular device through the access catheter.

19. A method as in claim 18, wherein the intravascular device is a stent retriever.
